SEQUENCE LISTING (1 sequence)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

(19) United States
(12) Patent Application Publication
(10) Pub. No.: US 2015/0209379 A1
(43) Pub. Date: Jul. 30, 2015

(54) TREATMENT OF PROSTATE CANCER AND HEMATOLOGIC NEOPLASMS

(71) Applicant: THOMAS JEFFERSON UNIVERSITY, PHILADELPHIA, PA (US)

(72) Inventors: Marja Tuuli Nevalainen, Gladwyne, PA (US); Vincent C. O. Njar, Glen Burnie, MD (US); Zhiyong Liao, Wynnewood, PA (US); Allen B. Reitz, Lansdale, PA (US); Mark E. McDonnell, Lansdale, PA (US)

(73) Assignee: THOMAS JEFFERSON UNIVERSITY, PHILADELPHIA, PA (US)

(21) Appl. No.: 14/421,608

(22) PCT Filed: May 10, 2013
(86) PCT No.: PCT/US13/40477

§ 371 Date: Feb. 13, 2015

Related U.S. Application Data
(60) Provisional application No. 61/683,901, filed on Aug. 16, 2012.

(51) Int. Cl. A61K 31/7076; A61K 45/06; A61K 31/7064

(52) U.S. Cl. CPC A61K 31/7076; A61K 31/7064; A61K 45/06

(57) ABSTRACT

Prostate cancer and hematological neoplasms are treated by administration of (i) a compound of Formula I: wherein: R¹ is —OH or —O—P(O)(OH)₂; and R² is (II) or (III); (ii) N⁶-benzyladenosine, (iii) N-(phenylmethyl)-7-β-D-ribofuranosyl-7H-pyrrolo [2,3 -d]pyrimidin-4-amine, (iv) N-(phenylmethyl)-7β-D-ribofuranosyl-7H-pyrrolo[2,3-d]pyrimidin-4-amine 5′-monophosphate; or a pharmaceutically acceptable salt thereof.

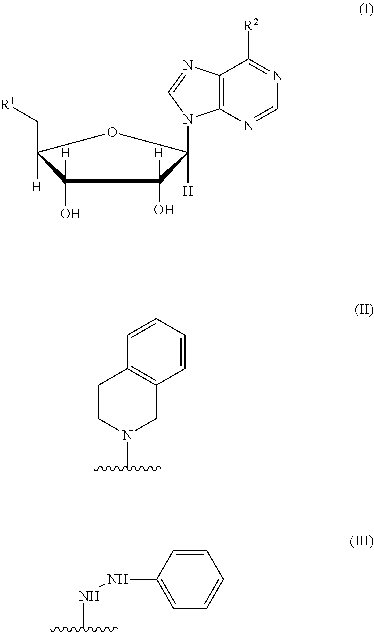

### CROSS-REFERENCE TO RELATED APPLICATION

The benefit of the filing date of U.S. Provisional Patent Application No. 61/683,901, filed Aug. 16, 2012, is hereby claimed. The entire disclosure of the aforesaid application is incorporated herein by reference.

### SEQUENCE LISTING

The instant application contains a Sequence Listing which has been submitted in ASCII format via EFS-Web and is hereby incorporated by reference in its entirety. Said ASCII copy, created on May 7, 2013, is named 37075_{—}0282_{—}00_WO_SL.txt and is 10,142 bytes in size.

### FIELD OF THE INVENTION

The invention relates to the treatment of prostate cancer and hematological neoplasms.

### BACKGROUND OF THE INVENTION

### Prostate Cancer

Prostate cancer is the third leading cause of cancer deaths among men in the United States. The number of new cases of prostate cancer, estimated at more than 220,000 per year in 2005, is expected to increase to more than 380,000 by 2025 because of the aging male population (Scardino, N Engl J Med 2003, 349:297-299). Organ-confined primary prostate cancer is treated by surgery, radiation, hormone therapy, or combinations of these treatment modalities, depending on the age, operability of the patient and tolerance for the specific treatment-related side-effects. Duration of response to hormonal therapy is limited and prostate cancers inevitably become castration-resistant and metastatic, a stage of the disease for which there is no curative treatment. For a significant fraction of prostate cancers, the existing therapies only provide a temporary relief of the symptoms, while the castration-resistant and/or metastatic forms of prostate cancer develop. Currently, there are no effective pharmacological therapies for advanced prostate cancer (Pestell R G, Nevalainen, M. T.: Prostate Cancer: Signaling Networks, Genetics and New Treatment Strategies. Totowa, Human Press, 2008).

Stat5 is one of 7 members of the Stat (Signal Transducer and Activation of Transcription) family of transcription factors in mammals, and consists of two distinct, but highly homologous, proteins, the 94-kDa Stat5a and 92-kDa Stat5b factors (Ihle et al., Curr Opin Cell Biol 2001, 13:211-217). The isoforms are encoded by separate genes (Id.) Stat5a and Stat5b (hereafter referred to collectively as “Stat5a/b”) are latent cytoplasmic proteins that act as both cytoplasmic signaling proteins and nuclear transcription factors. Stat5a and Stat5b become activated by phosphorylation on residue Tyr694 and Tyr 699, respectively, in the C-terminal domain predominantly by Janus tyrosine kinase-2 (Jak2), which is preassociated with the cytoplasmic domain of the prolactin (Prl) receptor (Pr1R). After phosphorylation, Stat5a/b homo- or hetero-dimerize and translocate to the nucleus where they bind to specific Stat5a/b response elements of target gene promoters (Id.)

Stat5 proteins are composed of five structurally and functionally conserved domains. The N-terminal domain stabilizes interactions between two Stat5 dimers to form tetramers for maximal transcriptional activation of weak promoters (John et al., Mol Cell Biol 1999, 19:1910-1918). The coiled-coil domain facilitates protein-protein interactions (Chen et al., Cell 1998, 93:827-839; Becker et al., Nature 1998, 394:145-151). The DNA-binding domain recognizes members of the GAS family of enhancers (Levy et al., Nat Rev Mol Cell Biol 2002, 3:651-662). The SH2 domain contains a binding pocket for the phosphotyrosine residue of another Stat5 molecule thereby mediating both receptor-specific recruitment followed by phosphorylation and STAT dimerization (Kisseleva et al., Gene 2002, 285:1-2425). The carboxy terminus carries a transactivation domain (TAD), which binds critical co-activators and is directly involved in initiation of transcription (Levy et al., supra; Darnell, Science 1997, 277:1630-1635).

Stat5a/b is critical for the viability of Stat5a/b-positive human prostate cancer cell lines in culture and for growth of human prostate cancer xenograft tumors in nude mice (Ahonen et al., J Biol Chem 2003, 278:27287-27292; Dagvadorj et al., Endocrinology 2007, 148:3089-3101; Dagvadorj et al., Clin Cancer Res 2008, 14:1317-1324). Adenoviral expression of a dominant-negative (DN) mutant of Stat5a, blocking both Stat5a and Stat5b, induced massive and rapid apoptotic death of human prostate cancer cells in culture. Id. Inhibition of Stat5a/b by antisense oligo-nucleotides or RNA interference also induced rapid apoptotic death of prostate cancer cells (Dagvadorj et al. Clin Cancer Res, supra), blocked human prostate cancer xenograft tumor growth (both subcutaneous and orthotopic) in nude mice, and down-regulated Bc1XL and Cyclin-D1 protein levels in prostate cancer cells (Id.).

Nuclear Stat5 in prostate cancer correlates with loss of differentiation. The levels of active Stat5a/b are increased in human prostate cancer but not in normal human prostate epithelium (Ahonen et al., supra). The levels of active Stat5a/b are elevated in prostate cancers of high histological grades (n=114, P<0.0001) (Li et al., Cancer Res 2004, 64:4774-4782), a finding later confirmed in a second independent study using material of 357 human prostate cancers (P=0.03) (Li et al., Clin Cancer Res 2005 11:5863-8). Active Stat5a/b levels are also elevated in the majority of castration-resistant recurrent human prostate cancers (Tan et al., Cancer Res 2008, 68:236-248). Increased active Stat5a/b in primary prostate cancer predicted early disease recurrence after initial treatment by radical prostatectomy (Li et al., Clin Cancer Res 2005, supra). When only prostate cancers of intermediate Gleason grades were analyzed, increased active Stat5a/b remained an independent predictive marker of early recurrence of prostate cancer (Id.).

Stat5a/b activation is strongly associated with high histological grade of prostate cancer (Li et al., Cancer Res 2004, 64:4774-4782; Li et al., Clin Cancer Res 2005, 11:5863-5868), but Stat5a/b is not active in normal prostate epithelium (Ahonen et al., supra). Stat5a/b activation in primary prostate cancer predicts early disease recurrence (Li et al., Clin Cancer Res 2005, 11:5863-5868). Nuclear Stat5a/b is over-expressed in castration-resistant clinical prostate cancers (Tan et al., Cancer Res 2008, 68:236-248; Tan et al., Endocr Relat Cancer 2008, 15:367-390). Stat5a/b is active in 95% of clinical hormone-refractory human prostate cancers, and synergizes with androgen receptor (AR) in prostate cancer cells (Tan et al., Cancer Res 2008, supra).

Stat5 is involved in induction of metastatic behavior of human prostate cancer cells. Nuclear Stat5 levels are increased in 61% of distant metastases of clinical prostate cancer (Gu et al., Endocrine-Related Cancer 2010, 17(2):481-493). Stat5 increased metastases formation of prostate cancer cells to the lungs of nude mice by 11-fold in an experimental in vivo metastases assay (Id.). Active Stat5 induced migration and invasion of prostate cancer cells, which was accompanied by Stat5-induced re-arrangement of the microtubule network. Active Stat5 expression was associated with decreased cell-surface E-cadherin levels, while heterotypic adhesion of prostate cancer cells to endothelial cells was stimulated by active Stat5 (Id.)

US Pat. Pub. 2007/0010468A1 describes methods and compositions for the inhibition of Stat5 in prostate cancer, and describes the treatment of prostate cancer by inhibition of Stat5. Transfection of the androgen-independent human prostate cell line CWR22Rv with an adenovirus vector carrying a dominant-negative Stat5a gene (AdNStat5) is described. A dose-dependent effect of the expression of said DNStat5 on prostate cell viability was observed. Microscopic assessment of the effect of AdDNStat5 on CWR22Rv cell viability confirmed extensive cell death following expression of DNStat5. AdDNStat5 also induced cell death in the androgen-sensitive human prostate cancer cell line, LnCap. Apoptotic cell death of prostate cancer cells expressing DNStat5 was verified by DNA fragmentation analysis and cell cycle analysis. Importantly, Stat inhibition kills both AR-positive and AR-negative prostate cancer cells indicating that both AR-independent and AR-associated pathways mediate the effects of Stat on prostate cancer cell viability (Gu et al., Am. J. Pathology 2010, 176(4):1959-1972).

Thus, blocking Stat5 activity was observed to induce apoptosis of prostate cancer cells.

While US Pat. Pub. 2007/0010468A1 and other sources discussed above suggest that interfering with the biological activity of Stat5 in human prostate is a therapeutic approach, what is needed is a small molecule that would be effective in inhibiting Stat5 activation and its biological activities, to inhibit growth of prostate tumor cells.

Hematological Neoplasms and Resistance to Treatment with ATP-Competitive Kinase Inhibitors

Hematological neoplasms, also known as hematological malignancies, comprise cancers that affect blood, bone marrow, and lymph nodes. They may be driven by chromosomal translocations. Hematological neoplasms may derive from myeloid or lymphoid cell lineages. Lymphomas, lymphocytic leukemias, and myeloma are of lymphoid origin. Acute and chronic myelogenous leukemia (also known and myeloid leukemia), myelodysplastic syndromes and myeloproliferative diseases are myeloid in origin. Hematological neoplasms include the following specific disorders, for example: Acute lymphoblastic leukemia (ALL), a cancer of the white blood cells characterized by excess lymphoblasts; acute myelogenous leukemia (AML), a cancer of the myeloid line of blood cells, characterized by the rapid growth of abnormal white blood cells; acute monocytic leukemia (AMoL, or AML-M5), which is considered a type of AML; chronic lymphoid leukemia (CLL), also known as B-cell chronic lymphocytic leukemia (B-CLL), a cancer that affects B cell lymphocytes; chronic myelogenous (or myeloid) leukemia (CML), also known as chronic granulocytic leukemia (CGL), a cancer of the white blood cell; Hodgkin's lymphoma; non-Hodgkin lymphomas (NHLs), which comprise a group of blood cancers that include any type of lymphoma except Hodgkin's lymphomas; and myelomas, such as multiple myeloma, which is a cancer of plasma cells.

CML is associated with a specific chromosomal abnormality, namely the Philadelphia or Ph¹ chromosome, which results from the translocation of the proto-oncogene c-abl from the long arm of chromosome 9 to the breakpoint cluster region (bcr) on chromosome 22, resulting in the formation of bcr-abl hybrid genes. The c-abl proto-oncogene normally encodes a protein (ABL) having tyrosine kinase activity. In cells carrying bcr-abl hybrid genes, the oncogene BCR-ABL is expressed. The tyrosine kinase activity of BCR-ABL is substantially augmented compared to the tyrosine kinase activity of ABL, and drives the disorders CML, AML and ALL.

The BCR-ABL fusion protein found in Philadelphia chromosome CML patients is a protein of approximately 210 kilodaltons (p210wt or p210BCR-ABL). The fusion transcript typically results from bcr exon 13 or 14 joined to abl exon 2. (Chissoe et al (1995) Genomics 27:67-82). The ABL portion of the p2lOwt therefore consists of exons 2-11 of the c-abl gene. The common convention in the literature to identify positions in the ABL tyrosine kinase portion in a particular BCR-ABL allele is to use the amino acid numbering for ABL which results from alternative splicing using exon 1 a, See GenBank protein ID AAB60394.1. Therefore the mutations referenced herein are likewise keyed to the amino acid numbering of this splice variant whose sequence is provided as ID AAB60394.1 and SEQ ID NO:1.

The first 26 amino acids of SEQ ID NO: 1 are encoded by abl exon la and are not found in the BCR-ABL fusion protein p210wt. The amino acid sequence encoded by exon 2 starts with E27 of proto-oncogene abl which results from the transcript which results form the alternative splicing that uses exon 1a. The protein kinase domain is from approximately Ile 242 to amino acid 493. In this numbering system, the phosphate-binding loop (p-loop) is amino acids 249-256, the catalytic region is amino acids 361-367 and the activation loop is amino acids 380-402.

Protein kinases such as ABL and BCR-ABL regulate cell proliferation. Inhibition of protein kinases has been accomplished by administration of ATP-competitive small molecules that block kinase enzymatic activity and thereby interfere with phosphorylation of cellular substrates. Examples of ATP-competitive small molecule inhibitors of kinases include, for example, PD180970, BMS-354825, imatinib, SU5416, SU6668, SU11248, AP23464, gefitinib, erlotinib, PD153035, and SB203580, the structures of which are shown in WO 2006/074149, Table 1.

The ATP-competitive tyrosine kinase inhibitor imatinib is highly effective in the treatment of hematologic neoplasms, particularly CML, AML and ALL. However, ATP-competitive kinase inhibitors such as imatinib have been shown to create selective pressures in target proliferating cells associated with the disorders for which the inhibitors are therapeutically administered. These selective pressures often result in the development of resistance in the target cells. Resistance may arise from mutation of the targeted kinase. Thus, a portion of imatinib-treated patients treated relapse when imatinib-resistant cells emerge.

Imatinib resistance may result from kinase mutations at the site of imatinib binding. Mutations may occur in the kinase catalytic domain, which includes the so-called ‘gatekeeper’ residue, Thr315, and other residues that contact imatinib during binding (e.g., Phe317 and Phe 359). Other mutations occur in the ATP binding domain (the p-loop) and in the activation loop, an area of the kinase structure involved in a conformational change that occurs upon imatinib binding.

Imatinib binding to BCR-ABL involves formation of a hydrogen bond to the Thr315 hydroxyl group. The substitution of isoleucine for threonine at position 315 is one of the most frequent BCR-ABL mutations in imatinib-resistant CML. Alteration of Thr315 to the larger and non-hydrogen bonding isoleucine directly interferes with imatinib binding. Thr315, though necessary for binding of imatinib, is not required for ATP binding to BCR-ABL. Thus, the catalytic activity, and therefore the tumor-promoting function of the BCR-ABL oncoprotein, is preserved in the T315I mutant.

Imatinib binding is also affected by mutations in the kinase p-loop. BCR-ABL sequence analysis in relapsed CML and Ph+ALL patients has detected p-loop mutations at Tyr253 and Glu255. Amino-acid substitutions at these positions may interfere with the distorted p-loop conformation required for imatinib binding. This is consistent with the observed in vivo resistance and the particularly poor prognosis of patients affected by BCR-ABL p-loop mutations such as Y253F and E255K (Branford et al., Blood, 102, 276-283 (2003).

Imatinib binding is associated with the inactive, unphosphorylated state of the BCR-ABL activation loop. Mutations in this region, such as H396R, destabilize a closed conformation of the activation loop and thereby counteract imatinib inhibition.

Another group of point mutations, remote from the imatinib binding site, lie in the carboxy-terminal lobe of the kinase domain. The most frequently detected BCR-ABL mutation falling into this group is M351T. The M351T mutation accounts for 15-20% of all cases of imatinib clinical resistance (Hochhaus et al., Leukemia, 18, 1321-31 (2004). M351T mutation appears to affect the precise positioning of residues in direct contact with imatinib (Cowan-Jacob et al., Mini Rev. Med. Chem., 4, 285-299 (2004), and Shah et al., Cancer Cell, 2, 117-125 (2002).

The above BCR-ABL mutations account for most of the BCR-ABL mutations which have been associated with imatinib resistance. However, a saturating mutational analysis of full-length BCR-ABL combined with a cellular screening procedure selecting for BCR-ABL-driven cell proliferation in the presence of imatinib, has revealed that mutations outside of the kinase domain can weaken kinase interaction with imatinib and thereby contribute to target resistance (Azam, et al., Cell, 112, 831-843 (2003).

BCR-ABL mutations clinically relevant to development of resistance to ATP-competitive kinase inhibitors such as imatinib include the following: G250E, F317L, Y253F, H396R, F311L, M351T, T3151, H396P, E255V, Y253H, Q252H, M244V, L387M, E355G, E255K and F359V. See, von Bubnoff et al., Leukemia 17, 829-838 (2003); Cowan-Jacob, et al., Mini Rev. Med. Chem. 4, 285-299 (2004); Hochhaus, et al., Leukemia 18, 1321-1331 (2004); Nardi, et al., Curr. Opin. Hematol. 11, 35-43 (2004); Ross, et al., Br. J. Cancer 90, 12-19; and Daub et al., Nature Reviews Drug Discovery, 3, 1001-10 (2004).

As of 2009, hematological malignancies accounted for 9.6% of all cancers diagnosed in the United States (“Facts & Statistics”, The Leukemia and Lymphoma Society). Needed are additional agents for treatment of hematological neoplasms, particularly hematological neoplasms that have become resistant to treatment with ATP-competitive small molecule inhibitors of kinases.

### SUMMARY OF THE INVENTION

Provided is a method of treating prostate cancer in a male in need of such treatment comprising administering to the male a therapeutically effective amount of one or more compounds of Formula I:

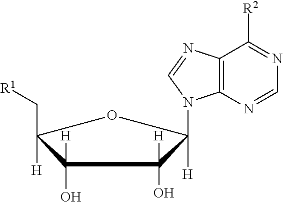

wherein:
R¹ is —OH or —O—P(O)(OH)₂; and
R² is

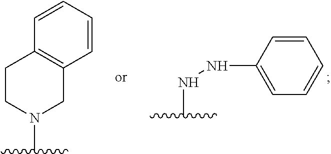 [SMILES: *N1CCc2ccccc2C1 ; *NNc1ccccc1]

or a pharmaceutically acceptable salt thereof.

Also provided is a method of treating prostate cancer in a male in need of such treatment comprising administering to the male a therapeutically effective amount of one or more of N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine-5′-monophosphate, or pharmaceutically acceptable salt thereof,

In certain embodiments, the compound is (2R,3R,4S,5R)-2-(6-(3,4-dihydroisoquinolin-2(1H)-yl)-9H-purin-9-yl)-5-(hydroxymethyptetrahydrofuran-3,4-diol or the 5′-monophosphate of (2R,3R,4S,5R)-2-(6-(3,4-dihydroisoquinolin-2(1H)-yl)-9H-purin-9-yl)-5-(hydroxymethyptetrahydrofuran-3,4-diol; or a pharmaceutically acceptable thereof.

In certain other embodiments, the compound is (2R,3S,4R,5R)-2-(hydroxymethyl)-5-(6-(2-phenylhydrazinyl)-9H-purin-9-yl)tetrahydrofuran-3,4-diol or the 5′-monophosphate of (2R,3S,4R,5R)-2-(hydroxymethyl)-5-(6-(2-phenylhydrazinyl)-9H-purin-9-yl)tetrahydrofuran-3,4-diol; or a pharmaceutically acceptable salt thereof.

In another embodiment, a method of inhibiting prostate cancer cell growth is provided, comprising contacting prostate cancer cells with an amount of (i) one or more compounds of Formula I, (ii) the compound N⁶-benzyladenosine; (iii) the compound N-(phenylmethyl)-7-β-D-ribofuranosyl-7H-pyrrolo[2,3-d]pyrimidin-4-amine, (iv) the compound N-(phenylmethyl)-7-β-D-ribofuranosyl-7H-pyrrolo[2,3-d]pyrimidin-4-amine-5′-monophosphate, or pharmaceutically acceptable salt of any of (i) through (iv), effective to inhibit such cell growth.

In another embodiment, a method of inhibiting prostate cancer cell growth in a male comprises administering to the male in need of treatment a therapeutically effective amount of (i) one or more compounds of Formula I, (ii) the compound N⁶-benzyladenosine; (iii) the compound N-(phenylmethyl)-7-β-D-ribofuranosyl-7H-pyrrolo[2,3-d]pyrimidin-4-amine, (iv) the compound N-(phenylmethyl)-7-β-D-ribofuranosyl-7H-pyrrolo[2,3-d]pyrimidin-4-amine-5′-monophosphate, or pharmaceutically acceptable salt of any of (i) through (iv), effective to inhibit such cell growth. The growth of prostate cancer cells in the male is inhibited by such administration.

The prostate cancer treated may be, for example, organ-confined primary prostate cancer, locally invasive advanced prostate cancer, metastatic prostate cancer, castration-resistant prostate cancer or recurrent castration-resistant prostate cancer. Metastatic prostate cancer is characterized by prostate cancer cells that are no longer organ-confined. Recurrent castration-resistant prostate cancer is prostate cancer that does not respond to androgen-deprivation therapy or prostate cancer that recurs after androgen-deprivation therapy.

In another embodiment, a method of treating prostate cancer in a male in need of such treatment is provided comprising administering to the male a therapeutically effective amount of (i) one or more compounds of Formula I, (ii) the compound N⁶-benzyladenosine; (iii) the compound N-(phenylmethyl)-7-β-D-ribofuranosyl-7H-pyrrolo[2,3-d]pyrimidin-4-amine, (iv) the compound N-(phenylmethyl)-7-β-D-ribofuranosyl-7H-pyrrolo[2,3-d]pyrimidin-4-amine-5′-monophosphate, or pharmaceutically acceptable salt of any of (i) through (iv), and at least one of (a) radiation therapy and (b) chemotherapy with an other chemotherapeutic agent effective against prostate cancer. By “other chemotherapeutic agent effective against prostate cancer” is meant a chemotherapeutic agent, other than a compound of Formula I, N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine-5′-monophosphate, or pharmaceutically acceptable salt thereof, that is effective in treating prostate cancer. The compounds (i)-(iv), and their pharmaceutically acceptable salts are referred to in this context as “primary anti-prostate cancer agent”. In some embodiments, the other chemotherapeutic agent is selected from the group consisting of docetaxel, mitoxantrone, estramustine, doxorubicin, etoposide, vinblastine, paclitaxel, carboplatin, and vinorelbine, and combinations thereof In some embodiments, the other chemotherapeutic agent is administered simultaneously with the said primary anti-prostate cancer agent. In other embodiments, the other chemotherapeutic agent is administered serially with said primary anti-prostate cancer agent. In one embodiment of simultaneous administration, the primary anti-prostate cancer agent and the other chemotherapeutic agent are contained in the same dosage form.

In some embodiments, the male treated according to the above methods is a male human being.

In another embodiment, a method for treatment of prostate cancer comprises administering to a male in need of such treatment a therapeutically effective amount of (A) (i) one or more compounds of Formula I, (ii) the compound N⁶-benzyladenosine; (iii) the compound N-(phenylmethyl)-7-β-D-ribofuranosyl-7H-pyrrolo[2,3-d]pyrimidin-4-amine, (iv) the compound N-(phenylmethyl)-7-β-D-ribofuranosyl-7H-pyrrolo[2,3-d]pyrimidin-4-amine-5′-monophosphate, or pharmaceutically acceptable salt of any of (i) through (iv), and (B) an androgen ablation therapy. In one embodiment, the androgen ablation therapy comprises androgen deprivation therapy. In another embodiment, the androgen ablation therapy comprises administration of at least one luteinizing hormone releasing hormone agonist, at least one anti-androgen, or at least one inhibitor of androgenic steroid synthesis in the prostate. In another embodiment, the androgen ablation therapy may comprise a combination of drugs from two or all three of the aforementioned categories. For example, the ablation therapy may comprise a combination of at least one luteinizing hormone releasing hormone agonist, and at least one anti-androgen, and/or at least one inhibitor of androgenic steroid synthesis.

In another embodiment, a compound (i), (ii), (ii) or (iii), or pharmaceutically acceptable salt thereof, is used for treating prostate cancer.

In one embodiment of each of the aforesaid anti-prostate cancer methods, the anti-prostate cancer agent administered (or primary anti-prostate cancer agent in the case of a combination therapy), is (2R,3R,4S, 5R)-2-(6-(3,4-dihydroisoquinolin-2(1H)-yl)-9H-purin-9-yl)-5-(hydroxymethyl)tetrahydrofuran-3,4-diol, or pharmaceutically acceptable salt thereof. In another embodiment, the anti-prostate cancer agent is the 5′-monophosphate of (2R,3R,4S,5R)-2-(6-(3,4-dihydroisoquinolin-2(1H)-yl)-9H-purin-9-yl)-5-(hydroxymethyl)tetrahydrofuran-3,4-diol. In another embodiment, the anti-prostate cancer agent is (2R,3S,4R,5R)-2-(hydroxymethyl)-5-(6-(2-phenylhydrazinyl)-9H-purin-9-yl)tetrahydrofuran-3,4-diol, or pharmaceutically acceptable salt thereof. In another embodiment, the anti-prostate cancer agent is the 5′-monophosphate of (2R,3S,4R,5R)-2-(hydroxymethyl)-5-(6-(2-phenylhydrazinyl)-9H-purin-9-yl)tetrahydrofuran-3,4-diol, or pharmaceutically acceptable salt thereof.

A method for treating a hematological neoplasm comprises administering to a subject in need of such treatment an effective amount of (a) one or more compounds of Formula I, (b) the compound N-(phenylmethyl)-7-β-D-ribofuranosyl-7H-pyrrolo[2,3-d]pyrimidin-4-amine, (c) the compound N-(phenylmethyl)-7-β-D-ribofuranosyl-7H-pyrrolo[2,3-d]pyrimidin-4-amine-5′-monophosphate, or pharmaceutically acceptable salt of any of (a), (b) or (c). In certain embodiments, the hematologic neoplasm is chronic myeloid leukemia, acute lymphoblastic lymphoma or acute myelogenous leukemia.

In another embodiment, one of more compounds of (a), (b) or (c), or pharmaceutically acceptable salt thereof, is used for treating a hematological neoplasm.

In some embodiments, the method further comprises administering to the individual being treated at least one other chemotherapeutic agent effective against hematologic neoplasms. By “other chemotherapeutic agent effective against hematological neoplasms” is meant a chemotherapeutic agent, other than (a) a compound of Formula I, (b) the compound N-(phenylmethyl)-7-β-D-ribofuranosyl-7H-pyrrolo[2,3-d]pyrimidin-4-amine, (c) the compound N-(phenylmethyl)-7-β-D-ribofuranosyl-7H-pyrrolo[2,3-d]pyrimidin-4-amine-5′-monophosphate, or a pharmaceutically acceptable salt thereof. The compounds of (a), (b) and (c) and their pharmaceutically acceptable salts are referred to in this context as “primary anti-hematological neoplasm agent”. In some embodiments, the other chemotherapeutic agent is administered serially with the primary anti-hematological neoplasm agent. In some embodiments, the agents are administered simultaneously. In some embodiments, they are administered in the same dosage form.

In some embodiments, the hematological neoplasm treated is a hematological neoplasm resistant to treatment with an ATP-competitive inhibitor of BCR-ABL, which resistance results from a mutation of one or more amino acid residues of BCR-ABL.

According to some embodiments of the method for treating a hematological neoplasm resistant to treatment with an ATP-competitive inhibitor of BCR-ABL, the ATP-competitive inhibitor of BCR-ABL to which the neoplasm has become resistant, is imatinib.

In some embodiments, the resistance to treatment with an ATP-competitive inhibitor of BCR-ABL results from a mutation in said BCR-ABL which comprises an alteration of at least one amino acid residue within the BCR-ABL p-loop. In some embodiments, the mutation comprises an alteration of amino acids Tyr 253 or Glu255.

In some embodiments, the resistance to treatment with an ATP-competitive inhibitor of BCR-ABL results from a mutation in said BCR-ABL which comprises an alteration of at least one amino acid residue within the BCR-ABL activation loop. In some embodiments, the mutation comprises an alteration of amino acid His396.

In some embodiments, the resistance to treatment with an ATP-competitive inhibitor of BCR-ABL results from a mutation in said BCR-ABL which comprises at least one mutation selected from the group consisting of F317L, H396R, M351T, H396P, Y253H, M244V, E355G, F359V, G250E, Y253F, F311L, T315I, E255V, Q252H, L387M, and E255K.

### Abbreviations

The following abbreviations may be utilized in the text and the figures:

N6BA or N⁶BA: N⁶-benzyladenosine.

NBPP: N-(phenylmethyl)-7-β-D-ribofuranosyl-7H-pyrrolo[2,3-d]pyrimidin-4-amine.

NBPPP: N-(phenylmethyl)-7-β-D-ribofuranosyl-7H-pyrrolo[2,3-d]pyrimidin-4-amine-5′-monophosphate.

### BRIEF DESCRIPTION OF THE FIGURES

FIG. 1A shows the effect of varying concentration of (2R,3R,4S,5R)-2-(6-(3,4-dihydroisoquinolin-2(1H)-yl)-9H-purin-9-yl)-5-(hydroxymethyptetrahydrofuran-3,4-diol (Compound 1) on the transcriptional activity of Stat5a and Stat5b on the (3-casein gene promoter in a luciferase reporter gene assay in the human prostate cancer cell line PC-3. FIG. 1B shows the activity of (2R,3S,4R,5R)-2-(hydroxymethyl)-5-(6-(2-phenylhydrazinyl)-9H-purin-9-yptetrahydrofuran-3,4-diol (Compound 2) in the same assay. Both compounds inhibited Stat5a and Stat5b transcriptional activity in a concentration-dependent manner.

FIG. 2 shows that Compound 2 inhibits phosphorylation of Stat5a in CWR22Rv1 human prostate cells after ligand (prolactin)-induced activation.

FIG. 3A show the results of a study demonstrating that Compound 1 blocks dimerization of Stat5a/b in human prostate cancer cells. FIG. 3B shows the activity of Compound 2 in the same assay. DMSO and the control compound C5 were included in the study as controls.

FIG. 4A shows a Western blot of lysates of K562 cells that were treated with Compound 1 or control. Western blotting of the lysates was carried out using the following as primary antibodies: anti-phosphotyrosine-Stat5a/b (“anti-pYStat5”) mAb or anti-Stat5ab mAb, to show the level of STAT5a/b phosphorylation in the treated cells. Anti-actin was included as a control. FIG. 4B shows the activity of Compound 2 in the same assay. FIGS. 4A and FIG. 4B demonstrate that Compounds 1 and 2 block phosphorylation of Stat5 in the human BCR-ABL-driven leukemia cell line K562.

FIG. 5A shows a Western blot of lysates of cells of the human BCR-ABL-driven chronic myeloid leukemia line KCL22S (imatinib-sensitive) and KCL22R (imatinib-resistant) that were treated with Compound 1 or control. FIG. 5B shows similar data for Compound 2. Both compounds blocked phosphorylation of Stat5 in both the imatinib-sensitive and imatinib-resistant cell lines.

FIG. 6A shows that Compound 1 reduces the number of viable CWR22Rv1 prostate cancer cells compared to the control compound, C5. FIG. 6B shows results with Compound 2 in the same assay.

FIG. 7A shows that Compound 1 reduces the number of viable K562 chronic myeloid leukemia cells compared to the control compound, C5. FIG. 7B shows results with Compound 2 in the same assay.

FIG. 8A shows that Compound 1 reduces the number of viable imatinib-sensitive KCL22 chronic myeloid leukemia cells compared to the control compound, C5. FIG. 8B shows results with Compound 2 in the same assay.

FIG. 9A shows that Compound 1 reduces the number of viable imatinib-resistant KCL22R chronic myeloid leukemia cells compared to the control compound, C5. FIG. 9B shows results with Compound 2 in the same assay.

FIG. 10 shows the effect of varying concentration of the compound N-(phenylmethyl)-7-β-D-ribofuranosyl-7H-pyrrolo[2,3-d]pyrimidin-4-amine (“NBPP”) in the transcriptional activity of Stat5 on the β-casein gene promoter in human prostate PC-3 cells in a luciferase reporter gene assay. The cells were transfected with pStat5a or pStat5b, pPr1R (prolactin receptor) plasmids, 0.5 μg of pBeta-casein-luc and 0.025 μg of pRL-TK (Renilla luciferase) plasmids as an internal control. The results, shown in FIG. 10, demonstrate that N-(phenylmethyl)-7-β-D-ribofuranosyl-7H-pyrrolo[2,3-d]pyrimidin-4-amine inhibits transcriptional activity of Stat5.

FIG. 11A comprises Western blots of lysates of K562 cells that were treated with compound NBPP or control compound at the indicated concentrations for 3 or 6 hours. Western blotting of the lysates was carried out using antibodies to phosphotyrosine-Stat5a/b (lanes marked “pStat5”) and Stat5ab (lanes marked “Stat5”). Anti-actin was included as a control. NBPP inhibited phosphorylation of Stat5 in the K562 cells.

FIG. 11B comprises Western blots of lysates of imatinib-sensitive (KCL22S) or imatinib-sensitive (KCL22R) cells that were treated with compound NBPP or control compound at the indicated concentrations for 6 or 16 hours. Western blotting of the lysates was carried out using antibodies to phosphotyrosine-Stat5a/b (lanes marked “pStat5”) and Stat5ab (lanes marked “Stat5”). Anti-actin was included as a control. NBPP inhibited phosphorylation of Stat5 in both the imatinib-sensitive and imatinib-resistant KCL22 cells.

FIG. 12 shows the result of a study demonstrating that NBPP blocks dimerization of Stat5a/b. DMSO, and the control compound C5 were included in the study.

FIGS. 13A-13D are a series of plots showing that compound NBPP reduces the number of viable CWR22Rv1 prostate cancer cells (13A), K562 leukemia cells (13C), imatinib-sensitive chronic myeloid leukemia cells (KCL22S), (13E) and imatinib-resistant KCL22 cells, (13F) vs. the control compound, C5, (13B, D, E, F).

FIG. 14A shows the effect of varying concentration of N⁶-benzyladenosine (N6BA), on the transcriptional activity of Stat5a and Stat5b on the β-casein gene promoter in a luciferase reporter gene assay. The compound inhibited Stat5 transcriptional activity in a concentration-dependent manner. FIG. 14B shows the results of a study demonstrating that N6BA blocks dimerization of Stat5a/b in human prostate cancer cells.

FIG. 15A shows that N6BA induces death of CWR22Rv1 prostate cancer cells vs. the control compound, C5 (FIG. 14B).

### DETAILED DESCRIPTION OF THE INVENTION

According to the present invention compounds of Formula I, or pharmaceutically acceptable salts thereof, inhibit the proliferation of prostate tumor cells and cells of hematological neoplasms, and cause their death, by inhibiting biological activities of Stat5a and/or Stat5b. According to another embodiment, the compounds N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine-5′-monophosphate, or pharmaceutically acceptable salt thereof, inhibit the proliferation of prostate tumor cells. According to another embodiment, the compounds N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine-5′-monophosphate, or pharmaceutically acceptable salt thereof, inhibit the proliferation of cells of hematological neoplasms, and cause their death, by inhibiting biological activities of Stat5a and/or Stat5b.

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

The expression “effective amount” or “therapeutically effective amount” when used to describe therapy to an individual suffering from prostate cancer or hematologic neoplasm, refers to the amount of a compound that inhibits the growth or proliferation of prostate cancer cells or cells of a hematological neoplasm, or alternatively induces apoptosis of such cells, preferably resulting in a therapeutically useful and preferably selective cytotoxic effect on prostate cancer cells. In one embodiment, the prostate cancer cells are part of a prostate tumor.

As used herein, the term “subject” or “patient” refers to any animal (e.g., a mammal), including, but not limited to humans, non-human primates, rodents, and the like. Typically, the terms “subject” and “patient” are used interchangeably herein in reference to a human subject.

The expression “kinase inhibitor” refers to an agent that acts to inhibit the kinase activity of a kinase.

The expression “ATP-competitive kinase inhibitor” means a kinase inhibitor that inhibits the kinase by competing with ATP for the ATP binding site on the kinase.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

(2R,3R,4S,5R)-2-(6-(3,4-Dihydroisoquinolin-2(1H)-yl)-9H-purin-9-yl)-5-(hydroxymethyl)tetrahydrofuran-3,4-diol (CAS Registry No. 402724-29-2) has the following chemical structure:

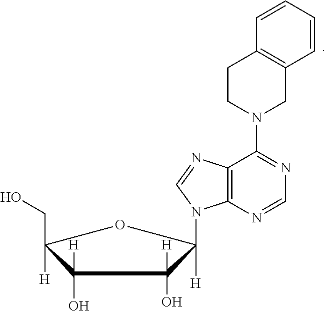

(2R,3R,4S,5R)-2-(6-(3,4-Dihydroisoquinolin-2(1H)-yl)-9H-purin-9-yl)-5-(hydroxymethyl)tetrahydrofuran-3,4-diol may be prepared according to Example 1.

(2R,3S,4R,5R)-2-(Hydroxymethyl)-5 -(6-(2-phenylhydrazinyl)-9H-purin-9-yl)tetrahydrofuran-3,4-diol (CAS Registry No. 35908-34-0) has the following chemical structure:

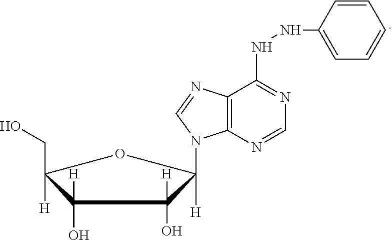

(2R,3S,4R,5R)-2-(Hydroxymethyl)-5-(6-(2-phenylhydrazinyl)-9H-purin-9-yl)tetrahydrofuran-3,4-diol may be prepared according to Example 2.

The 5′-monophosphates of the aforesaid compounds may be prepared according to known 5′-phosphorylation techniques.

N-(phenylmethyl)-7-β-D-ribofuranosyl-7H-pyrrolo[2,3-d]pyrimidin-4-amine has the following chemical structure:

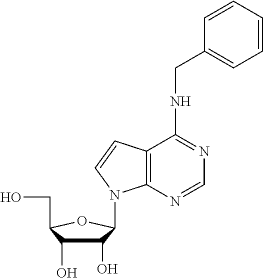

N-(phenylmethyl)-7-β-D-ribofuranosyl-7H-pyrrolo[2,3-d]pyrimidin-4-amine-5′-monophosphate has the following chemical structure:

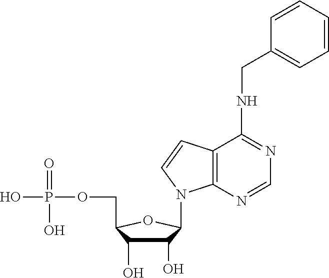

N-(phenylmethyl)-7-β-D-ribofuranosyl-7H-pyrrolo[2,3-d]pyrimidin-4-amine may be prepared from benzylamine and the known intermediate 4-chloro-7-β-D-ribofuranosyl-7H-Pyrrolo[2,3-d]pyrimidine, as described in Example 18. N-(phenylmethyl)-7-β-D-ribofuranosyl-7H-pyrrolo[2,3-d]pyrimidin-4-amine-5′-monophosphate may be prepared by phosphorylation of N-(phenylmethyl)-7-β-D-ribofuranosyl-7H-pyrrolo[2,3-d]pyrimidin-4-amine according to known 5′-phosphorylation techniques.

According to the present invention, it has been found that the above compounds block the transcriptional activity of Stat5a/b in human prostate cancer cells; blocks the dimerization of Stat5a/b in human prostate cancer cells; inhibit Stat5a/b phosphorylation in prostate cancer cells and in neoplastic cells of hematological neoplasms, particularly hematological neoplasms driven by expression of the oncogene BCR-ABL; and inhibit the viability and proliferation of such cells, and induces their apoptotic death. It has further been found that such effects are apparent even in cells of hematological neoplasm that display resistance to treatment with an ATP-competitive inhibitor of BCR-ABL, wherein the resistance results from a mutation of one or more amino acid residues of BCR-ABL.

In the activation cascade of Stat5a/b, the molecule first becomes phosphorylated at a conserved tyrosine residue in its C-terminus by an upstream tyrosine kinase (such as Jak2 or Src). Phosphorylation of Stat5a/b leads to dimerization of Stat5a/b, and the dimerized Stat5a/b translocates to the nucleus followed by binding of dimerized Stat5a/b to the promoters of its target genes to regulate transcription. Dimerization of Stat5a/b is required for Stat5a/b to bind DNA and exert is transcriptional activity. According to the present invention, the compounds described herein block Stat5a/b dimerization.

The compounds described herein are useful to provide therapy for primary, recurrent and metastatic prostate cancer. They may also be used for adjuvant therapy for prostate cancer after surgery and for sensitization of prostate cancer to radiation and chemotherapy, e.g., docetaxel chemotherapy. Certain of the compounds may be used for prevention of metastatic progression of the initial organ-confined primary prostate cancer after diagnosis and the initial treatment. They are also useful for treating recurrent castration-resistant prostate cancer and advanced disseminated prostate cancer.

Nuclear Stat5 levels are increased in 61% of distant metastases of clinical prostate cancer, and Stat5 promotes metastatic behavior of prostate cancer cells (Gu et al., Endocr Relat Cancer 2010; 17:481-493). Thus, the compounds described herein are useful in treating metastatic prostate caner, by inhibiting Stat5a/b activity.

During hormonal therapy, androgen-independent tumor cells eventually emerge, leading to clinical relapse, and the condition known as castration-resistant human prostate cancer. There are no effective treatments available for this condition. Stat5a/b is active in 95% of clinical castration-resistant human prostate cancers (Tan et al., Cancer Res 2008; 68(1):236-48). However, Stat5a/b has been shown to be active in 95% of clinical castration-resistant human prostate cancers (Tan et al., Cancer Res 2008; 68(1):236-48), thus presenting a target for therapy. Accordingly, another aspect of the present invention is the treatment of castration-resistant prostate cancer, by administration of certain compounds described herein, to a male in need of such treatment.

The compounds described herein may also be used for the treatment of hematologic neoplasms, of all types. Without limitation, the compounds described herein are particularly useful in the treatment of BCR-ABL-driven hematologic neoplasms, including without limitation CML and ALL. By “BCR-ABL-driven” is meant a neoplasm which is characterized by the presence of the BCR-ABL oncogene, as for example, in AML, ALL and CML.

Treatment of hematological neoplasm resistant to treatment with an ATP-competitive inhibitor of BCR-ABL may be carried out by following detection of BCR-ABL mutations in neoplastic cells of the subject. Mutations may be detected, for example, by sequencing the subject's bcr-abl gene in a leukemic cell, and comparing the sequence against a databank of resistance-conferring mutations. In addition to direct DNA sequencing, the following methods known to those skilled in the art may also be used to detect kinase mutations: Single-strand Conformation Polymorphism (SSCP); Denaturing Gradient Gel Electrophoresis (DGGE); Denaturing High-Performance Liquid Chromatography (DHPLC); Chemical Mismatch Cleavage (CMC); Enzyme Mismatch Cleavage (EMC); Heteroduplex analysis; and the use of DNA microarrays.

According to one embodiment of the invention, quantitative polymerase chain reaction (RQ-PCR) of BCR-ABL mRNA in imatinib-treated subjects may be used to detect patients at risk of resistance. A significant portion of imatinib-treated subjects displaying a two-fold or more increase in bcr-abl expression have detectable BCR-ABL mutations, indicating that such a rise in BCR-ABL may serve as a primary indicator to test patients for imatinib-deactivating BCR-ABL kinase domain mutations (Branford et al., Blood, 104, 2926-32 (2004)). Elevated BCR-ABL expression in the cells of a subject undergoing ATP-competitive kinase inhibitor therapy would suggest the need for mutation analysis to identify possible resistance-conferring BCR-ABL mutations, particularly in the BCR-ABL kinase domain.

According to some embodiments of the method for treating a hematological neoplasm resistant to treatment with an ATP-competitive inhibitor of BCR-ABL, the ATP-competitive inhibitor of BCR-ABL to which the neoplasm has become resistant is imatinib.

The resistant hematological neoplasm treated may result from a mutation in BCR-ABL which comprises an alteration of at least one amino acid residue within the BCR-ABL p-loop, e.g., amino acids Tyr 253 or G1u255. Alternatively, resistance may arise from an alteration of at least one amino acid residue within the BCR-ABL activation loop, e.g., an alteration of amino acid His396. In other embodiments, resistance may result from a mutation in BCR-ABL which comprises at least one mutation selected from the group consisting of F317L, H396R, M351T, H396P, Y253H, M244V, E355G, F359V, G250E, Y253F, F311L, T3151, E255V, Q252H, L387M, and E255K.

The compounds described herein may be converted to a salt for use according to the present invention. The term “pharmaceutically acceptable salt” refers to salts which possess toxicity profiles within a range that affords utility in pharmaceutical applications.

Suitable pharmaceutically-acceptable salts may take the form of base addition salts that may include, for example, metallic salts, e.g., alkali metal, alkaline earth metal and transition metal salts such as, for example, calcium, magnesium, potassium, sodium and zinc salts. The ammonium salt is a preferred salt.

Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. These salts may be prepared by conventional means from the subject compounds by reaction with the appropriate base.

The compounds may be administered by any route, including oral and parenteral administration. Parenteral administration includes, for example, intravenous, intramuscular, intraarterial, intraperitoneal, intranasal, rectal, intravesical, intradermal, topical or subcutaneous administration. Also contemplated within the scope of the invention is the instillation of drug in the body of the patient in a controlled formulation, with systemic or local release of the drug to occur at a later time. For example, the drug may be localized in a depot for controlled release to the circulation, or for release to a local site of tumor growth.

The specific dose of compound to obtain therapeutic benefit for treatment of a proliferative disorder will, of course, be determined by the particular circumstances of the individual patient including, the size, weight, age and sex of the patient, the stage of the disease, the aggressiveness of the disease, and the route of administration of the compound.

For example, a daily dosage of from about 0.01 to about 50 mg/kg/day may be utilized, or from about 1 to about 40 mg/kg/day, or from about 3 to about 30 mg/kg/day. Higher or lower doses are also contemplated. A therapeutically effective amount may also be estimated on the basis of the in vitro and in vivo studies hereinafter described.

The daily dose of the compound may be given in a single dose, or may be divided, for example into two, three, or four doses, equal or unequal, but preferably equal, that comprise the daily dose. When given intravenously, such doses may be given as a bolus dose injected over, for example, about 1 to about 4 hours.

The compounds may be administered in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The pharmaceutical composition is preferably in unit dosage form. In such form the preparation is divided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds described herein may be administered according to the invention in combination with one or more other active agents, for example, a least one other anti-proliferative compound, or drug to control side-effects, for example anti-emetic agents. The further active agent may comprise, for example a chemotherapeutic agent effective against prostate cancer or against hematological neoplasms. Such other agents for the treatment of prostate cancer include, for example, docetaxel, mitoxantrone, estramustine, doxorubicin, etoposide, vinblastine, paclitaxel, carboplatin, and vinorelbine.

Such other agents for the treatment of hematological neoplasms include, for example, targeted therapies such as imatinib, dasatinib, nilotinib or nelarabine. Further, such chemotherapeutic agentsfor the treatment of hematological neoplasms include vincristine, daunorubicine, idarubicine, cytarabine, L-asparaginase, etoposide, teniposide, 6-mercaptopurine, methotrexate, cyclophosphamide, prednisone, 6-thioguanine, hydroxyurea, ATRA, ATO, fludarabine, pentostatin, cladribine, chlorambucil, bendamustine, oxaliplatin, etoposide, topotecan, azacytidine, and decitabine. Monoclonal antibodies for the treatment of hematological nneoplasms include rituximab, alemtuzumab and ofatumumab.

In one embodiment of the invention, (i) a compound of Formula I, (ii) the compound N⁶-benzyladenosine; (iii) the compound N-(phenylmethyl)-7-β-D-ribofuranosyl-7H-pyrrolo[2,3-d]pyrimidin-4-amine, (iv) the compound N-(phenylmethyl)-7-β-D-ribofuranosyl-7H-pyrrolo[2,3-d]pyrimidin-4-amine-5′-monophosphate, or pharmaceutically acceptable salt of any of (i) through (iv), may be used to sensitize prostate cancer to radiation treatment and/or chemotherapy, e.g., docetaxel chemotherapy.

Radiation therapy uses high-energy rays or particles to kill cancer cells. The radiation treatment may comprise, for example, brachytherapy, i.e., implantation radiotherapy, or external beam radiation. Brachytherapy uses small radioactive pellets, or “seeds” implant radiation therapy or external-beam radiation therapy. Methods of external beam radiation and brachytherapy are well-known to those skilled in the art.

In one embodiment of the invention, an aforementioned primary active agent (i), (ii), (iii) or (iv), and an other anticancer agent, are co-formulated and used as part of a single pharmaceutical composition or dosage form. The compositions according to this embodiment of the invention comprise one or more of the aforementioned primary agents, and at least one other chemotherapeutic agent, in combination with a pharmaceutically acceptable carrier. In such compositions, the primary agent, and the second chemotherapeutic agent, may together comprise from 0.1 to 99.99 weight percent of the total composition. The compositions may be administered by any route and according to any schedule which is sufficient to bring about the desired antiproliferative effect in the patient.

According to other embodiments of the invention, the combination of the primary anticancer agent (i), (ii), (iii) or (iv), or pharmaceutically acceptable salt thereof, and the at least one other chemotherapeutic agent, may be formulated and administered as two or more separate compositions, at least one of which comprises the primary anticancer agent, and the other comprises the other chemotherapeutic agent. The separate compositions may be administered by the same or different routes, administered at the same time or different times, and administered according to the same schedule or on different schedules, provided the dosing regimen is sufficient to bring about the desired antiproliferative effect in the patient. When the drugs are administered in serial fashion, it may prove practical to intercalate administration of the two drugs, wherein a time interval, for example a 0.1 to 48 hour period, separates administration of the two drugs.

Prostate cancer cells, like certain normal prostate epithelial cells, can chronically depend on a critical level of androgenic stimulation for their net continuous growth and survival. Almost all prostate carcinomas are originally androgen-dependent. Androgen ablation has been used as a standard systemic therapy for metastatic prostate cancer. Androgen ablation is a type of therapy where the purpose is to remove or reduce the amount of androgen in a subject. Androgen ablation techniques for ablating serum androgens include, for example, androgen ablation by drug treatment, i.e., (i) treatment with luteinizing hormone releasing hormone (LHRH) agonists, e.g., goserelin or leuprolide, (ii) treatment with an anti-androgen such as flutamide or bicalutamide; or (iii) treatment with an agent that suppresses local synthesis of androgenic steroids in the prostate, e.g., the agent ketoconazole or abiraterone. Androgen ablation therapy can include a combination drug treatment including combining one or more drugs from two or three of the aforementioned categories. For example, at least one LHRH agonist may be combined with at least one anti-androgen and/or at least one inhbitor of prostate synthesis of androgenic steroids. Androgen ablation may also include castration in the form of surgical castration (orchiectomy, i.e., surgical removal of testes) or chemical castration. Androgen ablation therapy may include a combination of the drug-based therapy, described above, and castration.

Stat5a/b has been shown to synergize with androgen receptor (AR) in prostate cancer cells (Tan et al., Cancer Res 2008, 68:236-248). Specifically, active Stat5a/b increases transcriptional activity of AR, and AR, in turn, increases transcriptional activity of Stat5a/b. Liganded AR and active Stat5a/b physically interact in prostate cancer cells and, importantly, enhance nuclear localization of each other. This synergy between AR and the prolactin signaling protein Stat5a/b in human prostate cancer cells provides a further target for therapeutic intervention in the treatment of prostate cancer.

Accordingly, inhibition of Stat5a/b activity achieved with administration of one or more of a primary anticancer agents ((i) a compound of Formula I, (ii) the compound N⁶-benzyladenosine; (iii) the compound N-(phenylmethyl)-7-β-D-ribofuranosyl-7H-pyrrolo[2,3-d]pyrimidin-4-amine, or (iv) the compound N-(phenylmethyl)-7-β-D-ribofuranosyl-7H-pyrrolo[2,3-d]pyrimidin-4-amine-5′-monophosphate), or pharmaceutically acceptable salt thereof, when coupled with androgen ablation therapy, may lead to enhanced and synergistic inhibition of prostate cancer cell growth. Thus, according to another embodiment of the invention, one or more of the aforementioned primary anticancer agents is administered in conjunction with an androgen ablation therapy for treatment of prostate cancer. The androgen ablation therapy may comprise drug-based androgen ablation such as treatment with an LHRH agonist and/or anti-androgen, castration, or both. Where drug-based androgen ablation is employed, the primary anticancer agent may be combined with the androgen ablation agent in a single composition or dosage form, separated in two compositions or dosage forms, administered by the same or separate routes, and administered simultaneously or at different times.

The practice of the invention is illustrated by the following non-limiting examples.

### EXAMPLES

Cells used in the following procedures were cultured according to the following conditions. Human prostate cancer cell lines PC3 and CWR22Rv1 and leukemia cells (K562, KCL22, KCL22R) were cultured in RPMI 1640 containing penicillin/streptomycin and 10% fetal bovine serum (FBS), 0.5 nmol/L of dihydroestosterone (DHT) was additionally supplemented for LNCaP cells.

The following is the structure of the compound designated as “C5”, used as a control in certain of the following examples:

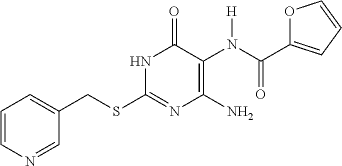 [SMILES: Nc1nc(SCc2cccnc2)[nH]c(=O)c1NC(=O)c1ccco1]

### Example 1

### Synthesis of (2R,3R,4S,5R)-2-(6-(3,4-Dihydroisoquinolin-2(1H)-yl)-9H-purin-9-yl)-5-(hydroxymethyl)tetrahydrofuran-3,4-diol (Compound 1)

1,2,3,4-Tetrahydro-isoquinoline (60 mg, 0.45 mmol, Aldrich), 6-chloropurine-9-β-D-ribofuranoside (100 mg, 0.35 mmol, Aldrich), diisopropylethylamine (193 μL, 1.05 mmol), and dimethylformamide (1 mL) were combined in a microwave tube (5 mL). The reaction was irradiated 15 minutes at 90° C. in the microwave. After cooling the reaction was filtered and then purified using reverse phase chromatography (gradient from 10% acetonitrile to 90% acetonitrile/water—both solvents containing 0.1% TFA). The appropriate fractions were combined and lyophilized to yield the title compound (70 mg, 52% yield. MS(ES⁺)=384(MH)⁺.

### Example 2

### Synthesis of (2R,3S,4R,5R)-2-(hydroxymethyl)-5-(6-(2-phenylhydrazinyl)-9H-purin-9-yl)tetrahydrofuran-3,4-diol (Compound 2)

Phenyl hydrazine (49 mg, 0.45 mmol, Aldrich), 6-chloropurine-9-β-D-ribofuranoside (100 mg, 0.35 mmol, Aldrich), diisopropylethylamine (193 μL, 1.05 mmol), and dimethylformamide (1 mL) were combined in a microwave tube (5 mL). The reaction was irradiated 15 minutes at 90° C. in the microwave. After cooling the reaction was filtered and then purified using reverse phase chromatography (gradient from 10% acetonitrile to 90% acetonitrile/ water—both solvents containing 0.1% TFA). The appropriate fractions were combined and lyophilized to yield the title compound (35 mg, 28% yield. MS(ES⁺)=359 (MH)⁺.

### Example 3

### Blockade of Stat5a Transcriptional Activity

Cells of the human prostate cell line PC-3 were plated into 96-well plate at the density of 2×10⁵ per well. After 24 hours of plating, cells were transiently co-transfected using FuGENE6 (Roche) with 0.25 μg of each of pStat5a, pPrlR (prolactin receptor) plasmids, 0.5 μg of pBeta-casein-luc and 0.025 μg of pRL-TK (Renilla luciferase) plasmids as an internal control. After another 24 hours of transfection, the cells were starved in serum-free medium for 8 hours, pretreated with (2R,3R,4S,5R)-2-(6-(3,4-dihydroisoquinolin-2(1H)-yl)-9H-purin-9-yl)-5-(hydroxymethyl)tetrahydrofuran-3,4-diol (Compound 1) or (2R,3S,4R,5R)-2-(hydroxymethyl)-5-(6-(2-phenylhydrazinyl)-9H-purin-9-yl)tetrahydrofuran-3,4-diol (Compound 2), for 1 hour, and then stimulated with 10 nM human prolactin (hPrl) in the serum-free medium for additional 16 hours. The lysates were assayed for firefly and Renilla luciferase activities using the Dual-Luciferase reporter assay system (Promega). The firefly luciferase activity was normalized to the Renilla luciferase activity of the same sample, and the mean was calculated from the parallels. From the mean values of each independent run, the overall mean and its standard deviation (S.D.) were calculated.

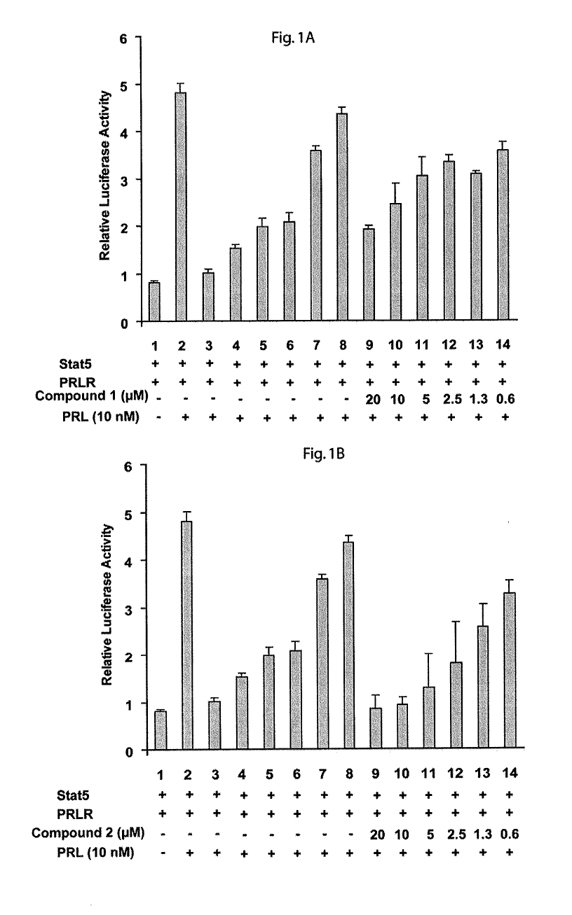

The results are shown in FIGS. 1A (Compound 1) and 1B (Compound 2). Both compounds effectively block the transcriptional activity of Stat5a in the PC-3 human prostate cancer cells.

### Example 4

### N⁶-Benzyladenosine-5′-Monophosphate Inhibition of Stat5 Phosphorylation in Prostate Cells

CWR22Rv1 cells were starved for 24 h in serum-free medium, treated with Compound 2 at 12, 25, 50 and 100 μM concentrations for 2 h, followed by stimulation with 10 nM Prl for 30 minutes. Stat5a was immunoprecipitated with anti-Stat5a pAb (4 gg/ml, Millipore, Billerica, Mass.). Immunoprecipitates of CWR22Rv1 cells were blotted with anti-phospho-Stat5ab mAb (1 μg/ml, Advantex BioReagents, Conroe, Tex.) or anti-Stat5a/b mAb (BD Biosciences, San Jose, Calif.). Whole cell lysates were immunoblotted with anti-actin pAb for loading control.

The protocol in more detail comprised the following. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Stat5a was immunoprecipitated from whole cell lysates with anti-Stat5a (4 μl/ml; Millipore, Billerica, Mass.) pAb. Antibody was captured by incubation for 60 min with protein A-Sepharose beads (Amersham Pharmacia Biotech, Piscataway, N.J.). For Western blotting, the primary antibodies were anti-phospho-Stat5ab mAb (1 μg/ml, Advantex BioReagents, Conroe, Tex.) or anti-Stat5a/b mAb (BD Biosciences, San Jose, Calif.). Other antibodies for Western blotting were anti-β-actin pAb (1:4,000; Sigma.

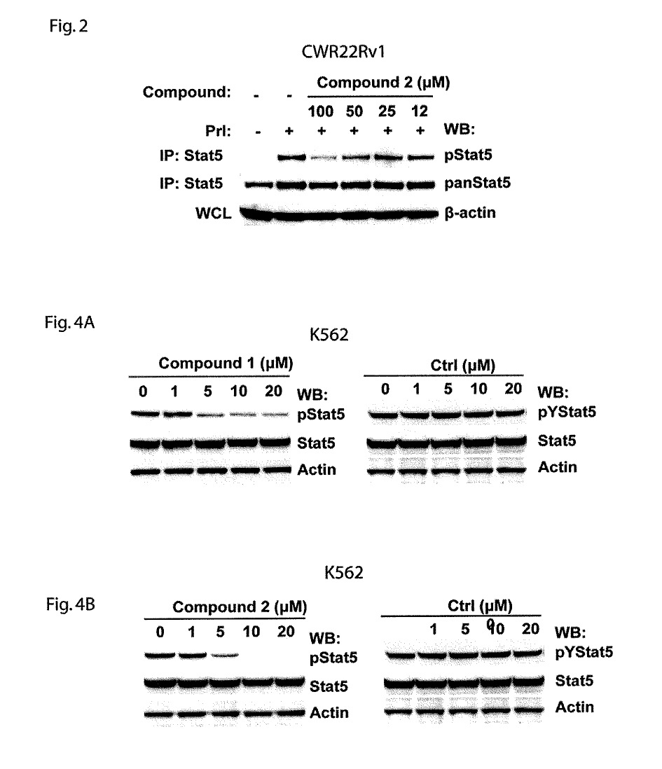

The results in FIG. 2 show that Compound 2 inhibits Stat5a phosphorylation in the human prostate cell line CWR22Rv1.

### Example 5

### Inhibition of Stat5 Dimerization Assay

Dimerization of Stat5a/b molecules is required for transcriptional activity of Stat5a/b and its biological effects. The following study demonstrates that Compounds 1 and 2 block dimerization of Stat5a/b in human prostate cancer cells.

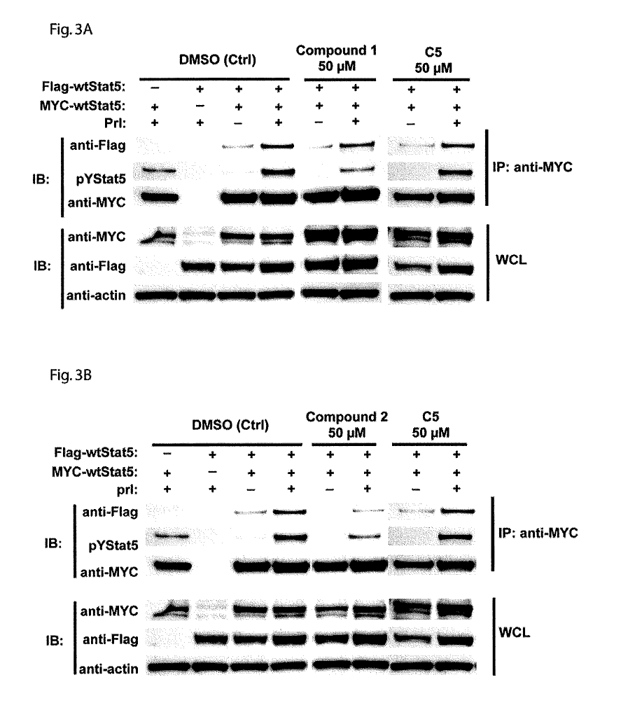

FLAG-tagged Stat5a and MYC-tagged Stat5a were generated by standard molecular biology cloning techniques. Plasmid pCMV3-FLAG-Stat5a, pCMV3-MYC-Stat5a and pPr1R were co-transfected using FuGENE6 (Roche) into PC-3 cells (2 μg of each plasmid per 1×10⁷ cells). The cells were starved for 20 hours, pretreated with test compound for 2 hours, then stimulated with hPrl (10 nM) in RPMI 1640 without serum for 30 minutes. The cell lysates were immunoprecipitated with 25 μl anti-FLAG M2 polyclonal affinity gel (2 μg/ml, Sigma), anti-MYC pAb (1 μg/ml, Upstate) or normal rabbit serum (NRS). The primary antibodies were used in the immunoblottings at the following concentrations: anti-FLAG pAb (1:1000; Stratagene), anti-MYC mAb (1:1000; Sigma), anti-Stat5a/b mAb (1:250) (Transduction Laboratories) detected by horseradish peroxidase-conjugated secondary antibodies. The results are shown in FIG. 3A for Compound 1 and FIG. 3B for Compound 2.

In each of FIGS. 3A and 3B, lane 1 demonstrates cells transfected only with MYC-tagged Stat5a (the third panel from the bottom). Lane 2 demonstrates cells transfected only with FLAG-tagged Stat5a (the second panel from the bottom). Lanes 3-8 demonstrate cells transfected with both FLAG-tagged Stat5a and MYC-tagged-Stat5a (the second and third panels from the bottom, respectively). Lanes 1, 2, 4, 6 and 8 represent cells stimulated with human prolactin (Prl) for 30 minutes which induces dimerization of Stat5. Two hours prior to Prl-stimulation, the cells had been treated with DMSO (lanes 1-4), the control compound C5 (lanes 7 and 8) and either Compound 1 (lanes 5 and 6 in FIG. 3A) or Compound 2 (lanes 5 and 6 in FIG. 3B). When MYC-tagged Stat5a was immunoprecipitated from the cells (the second panel from the top in each of FIGS. 3A and 3B) and immunoblotted with anti-FLAG pAb (top panel in each of FIGS. 3A and 3B) the control compound did not inhibit dimerization of Stat5a (lane 8). In contrast, both Compound 1 and 2 effectively inhibited Stat5a dimerization (lane 6) (=weak band in Prl-stimulated cells). It should be noted the drug effect in this study was not due to cell apoptosis induction, as the treatment time was only 2 hours. Treatment of at least 48 hours is required for the compound to induce prostate cancer cell apoptosis.

### Example 6

### Inhibition of Phosphorylation of Stat5 in K562 Cells

The following experiment demonstrates that Compounds 1 and 2 inhibits constitutive Stat5a/b phosphorylation in human chronic myeloid leukemia (K562) cells driven by BCR-ABL. Exponentially growing K562 cells were treated with 5 μM Compound 1, Compound 2 or control for 3 h after which the cells were harvested. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/mlaprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out using, as primary antibodies, the following antibodies at the following concentrations: anti-phosphotyrosine-Stat5a/b (Y694/Y699) mAb (1 μg/ml, Advantex BioReagents, Conroe, Tex.) or anti-Stat5ab mAb (1:250; BD Biosciences, San Jose, Calif.), anti-human Abl mAb (Calbiochem).

The results are shown in FIGS. 4A and 4B, demonstrating that Compounds 1 and 2 block phosphorylation of Stat5 in the human BCR-ABL-driven leukemia cell line K562.

### Example 7

### Inhibition of Phosphorylation in Imatinib-Sensitive and Imatinib-Resistant Human Leukemic Cells

The following experiment demonstrates that Compounds 1 and 2 inhibit constitutive Stat5a/b phosphorylation in human imatinib-sensitive (KCL22S) and imatinib-resistant (KCL22R) chronic myeloid leukemia cells driven by BCR-ABL. Exponentially growing KCL22S and KCL22R cells were treated with Compound 1 or Compound 2 at various concentrations for 3 h and 16 h (Compound 1) or 3 h, 6 h or 16 h (Compound 2) after which the cells were harvested. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out using, as primary antibodies, the following antibodies at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.) or anti-actin pAb (Sigma). Anti-actin was included as a control.

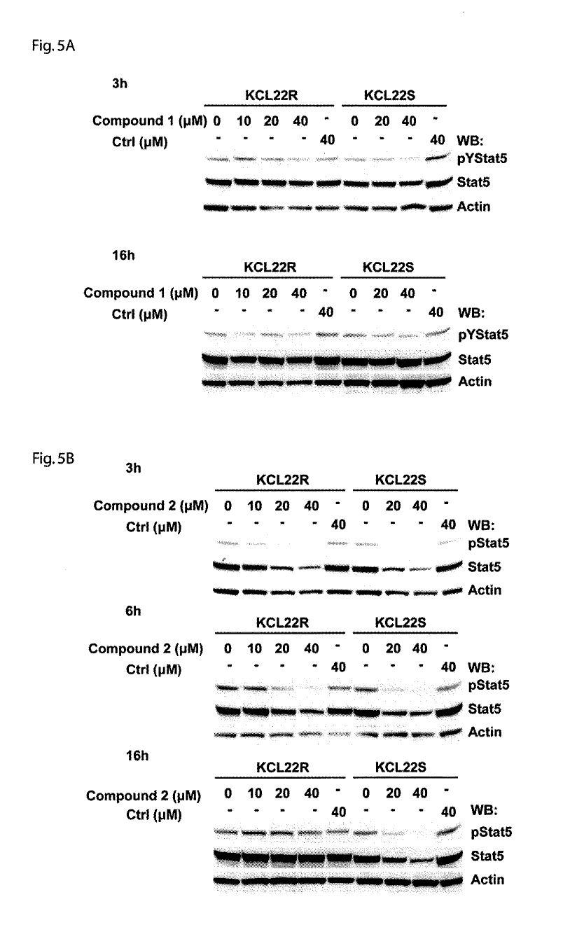

The results, shown in FIG. 5A (Compound 1) and 5B (Compound 2), demonstrate that the compounds block phosphorylation of Stat5 in the human BCR-ABL-driven parental KCL22 cells (KCL22S) and in imatinib-resistant KCL22 cells (KCL22R).

### Example 8

### Reduction in the Viability of CWR22Rv1 Human Prostate Cancer Cells

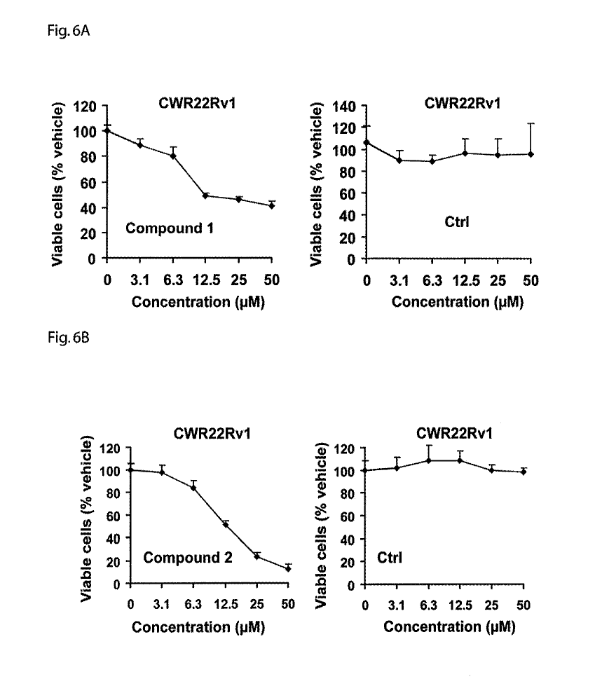

Exponentially growing CWR22Rvl prostate cancer cells were treated with Compound 1, Compound 2 or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). Compound 1 (FIG. 6A) and Compound 2 (FIG. 6B) decreased the number of viable prostate cancer cells compared to the control compound.

### Example 9

### Reduction in the Viability of K562 Human Chronic Myeloid Leukemia Cells

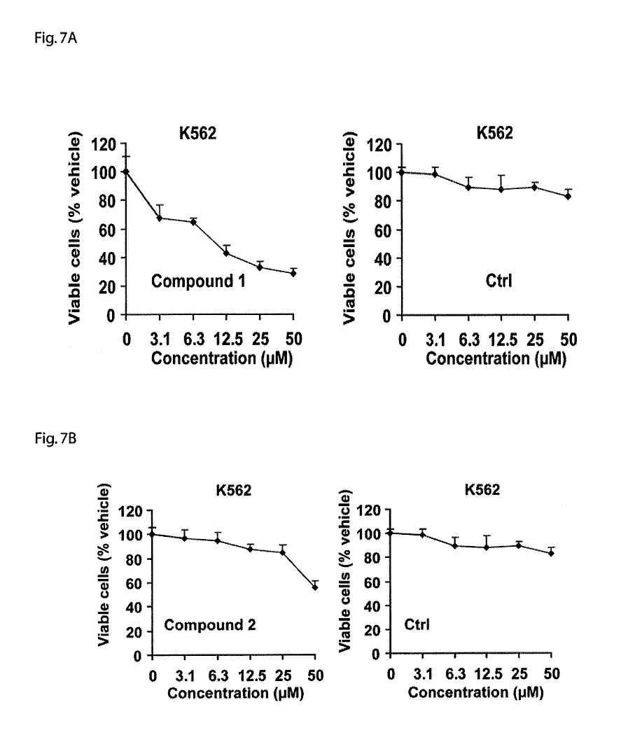

Exponentially growing K562 human chronic myeloid leukemia cells were treated with Compound 1, Compound 2 or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). Compound 1 (FIG. 7A) and Compound 2 (FIG. 7B) decreased the number of viable leukemic cells compared to the control compound.

### Example 10

### Reduction in the Viability of KCL22 Human Chronic Myeloid Leukemia Cells

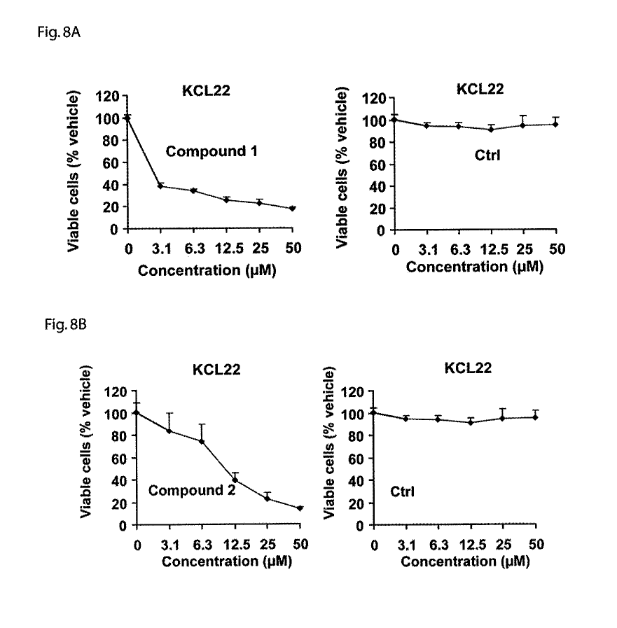

Exponentially growing KCL22 human chronic myeloid leukemia cells were treated with Compound 1, Compound 2 or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). Compound 1 (FIG. 8A) and Compound 2 (FIG. 8B) decreased the number of viable leukemic cancer cells compared to the control compound.

### Example 11

### Reduction in the Viability of KCL22R Imatinib-Resistant Human Chronic Myeloid Leukemia Cells

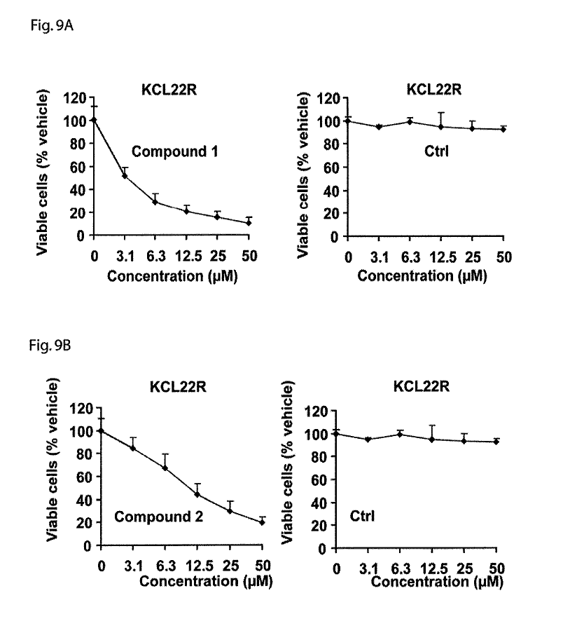

Exponentially growing KCL22R imatinib-resistant human chronic myeloid leukemia cells were treated with Compound 1, Compound 2 or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). Compound 1 (FIG. 9A) and Compound 2 (FIG. 9B) decreased the number of viable leukemic cancer cells compared to the control compound.

### Example 12

### N-(phenylmethyl)-7-β-D-ribofuranosyl-7H-pyrrolo[2,3-d]pyrimidin-4-amine Inhibition of Stats Transcriptional Activity

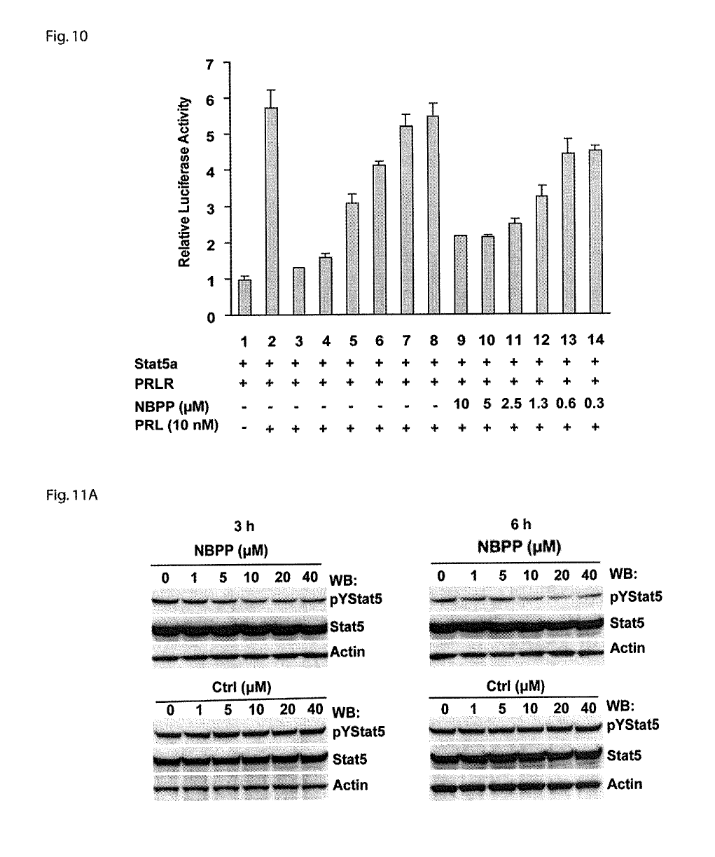

Cells of the human prostate cell line PC-3 were plated into 96-well plate at the density of 2×10⁵ per well. After 24 hours of plating, cells were transiently co-transfected using FuGENE6 (Roche) with 0.25 μg of pStat5a, pPrlR (prolactin receptor) plasmids, 0.5 μg of pBeta-casein-luc and 0.025 μg of pRL-TK (Renilla luciferase) plasmids as an internal control. After another 24 hours of transfection, the cells were starved in serum-free medium for 8 hours, pretreated with N-(phenylmethyl)-7-β-D-ribofuranosyl-7H-pyrrolo[2,3-d]pyrimidin-4-amine (“NBPP”) or the control compound C5, for 1 hour, and then stimulated with 10 nM human prolactin (hPrl) in the serum-free medium for additional 16 hours. The lysates were assayed for firefly and Renilla luciferase activities using the Dual-Luciferase reporter assay system (Promega). Three independent experiments were carried out in triplicate. The firefly luciferase activity was normalized to the Renilla luciferase activity of the same sample, and the mean was calculated from the parallels. From the mean values of each independent run, the overall mean and its standard deviation (S.D.) were calculated. The results, shown in FIG. 10, demonstrate that NBPP inhibits transcriptional activity of Stat5 with an IC50 of 1.6 μM.

### Example 13

### N-(phenylmethyl)-7-β-D-ribofuranosyl-7H-pyrrolo[2,3-d]pyrimidin-4-amine Inhibits Constitutive Stat5a/b Phosphorylation in K562 Cells and KCL22 Cells

Exponentially growing K562 cells were treated with NBPP for 3 h or 6 h after which the cells were harvested. Exponentially growing K562 cells or imatinib-resistant K562 cells were treated with NBPP for 6 h or 16 h after which the cells were harvested. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out using, as primary antibodies, the following antibodies at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.), anti-human Abl mAb (Calbiochem) or anti-phosphotyrosine mAb (Millipore). Anti-actin was included as a control.

The results for K562 cells are shown in FIG. 11A. The results for KCL22 cells (KCL22S) and imatinib-resistant KCL22 cells (KCL22R) are shown in FIG. 11B. The results demonstrate that NBPP inhibits phosphorylation of Stat5 in imatinib-sensitive leukemic cells, and also in imatinib-resistant leukemic cells.

### Example 14

### N-(phenylmethyl)-7-β-D-ribofuranosyl-7H-pyrrolo[2,3-d]pyrimidin-4-amine Blockage of Stat5 Dimerization

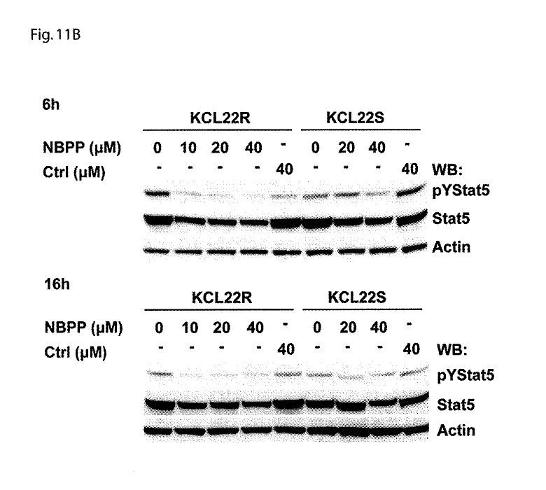

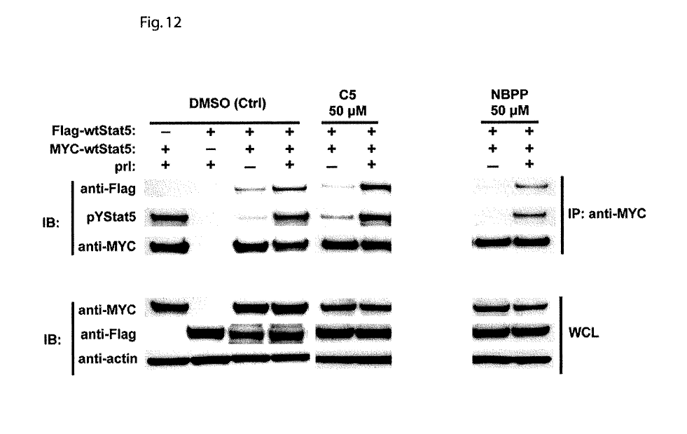

The following study demonstrates that Stat5 dimerization is blocked by NBPP. FLAG-tagged Stat5a and MYC-tagged Stat5a were generated by standard molecular biology cloning techniques. Plasmid pCMV3-FLAG-Stat5a, pCMV3-MYC-Stat5a and pPrlR were co-transfected using FuGENE6 (Roche) into PC-3 cells (2 μg of each plasmid per 1×10⁷ cells). The cells were starved for 20 hours, pre-treated with NBPP for 2 h, then stimulated with hPrl (10 nM) in RPMI 1640 without serum for 30 minutes. The cell lysates were immunoprecipitated with 25 μl anti-FLAG M2 polyclonal affinity gel (2 μg/ml, Sigma), anti-MYC pAb (1 μg/ml, Upstate) or normal rabbit serum (NRS). The primary antibodies were used in the immunoblottings at the following concentrations: anti-FLAG pAb (1:1000; Stratagene), anti-MYC mAb (1:1000; Sigma), anti-Stat5a/b mAb (1:250) (Transduction Laboratories) detected by horseradish peroxidase-conjugated secondary antibodies. The results are shown in FIG. 12.

Lane 1 of FIG. 12 demonstrates cells transfected only with MYC-tagged Stat5a (the third panel from the bottom). Lane 2 demonstrates cells transfected only with FLAG-tagged Stat5a (the second panel from the bottom). Lanes 3-8 demonstrate cells transfected with both FLAG-tagged Stat5a and MYC-tagged-Stat5a (the second and third panels from the bottom, respectively). Lanes 1, 2, 4, 6 and 8 represent cells stimulated with human prolactin (Prl) for 30 minutes which induces dimerization of Stat5. Two hours prior to Prl-stimulation, the cells had been treated with DMSO (lanes 1-4), the control compound C5 (lanes 5 and 6) or NBPP (lanes 7 and 8) to test whether the Stat5a/b-inhibitor compound, NBPP, would be able to inhibit dimerization of StatS. When MYC-tagged Stat5a was immunoprecipitated from the cells (the third panel from the top) and immunoblotted with anti-FLAG pAb (top panel) the control compound did not inhibit dimerization of Stat5a (lane 6). In contrast, NBPP (lane 8) effectively inhibited Stat5a dimerization (lane 8) (=very weak band in Prl-stimulated cells). It should be noted the drug effect in this study was not due to cell apoptosis induction, as the treatment time was only 2 hours. Treatment of at least 48 hours is required for the compound to induce prostate cancer cell apoptosis.

### Example 15

### N-(phenylmethyl)-7-β-D-ribofuranosyl-7H-pyrrolo[2,3-d]pyrimidin-4-amine Reduces the Viability of CWR22Rv1 Human Prostate Cancer Cells and K562 Human Leukemic Cells

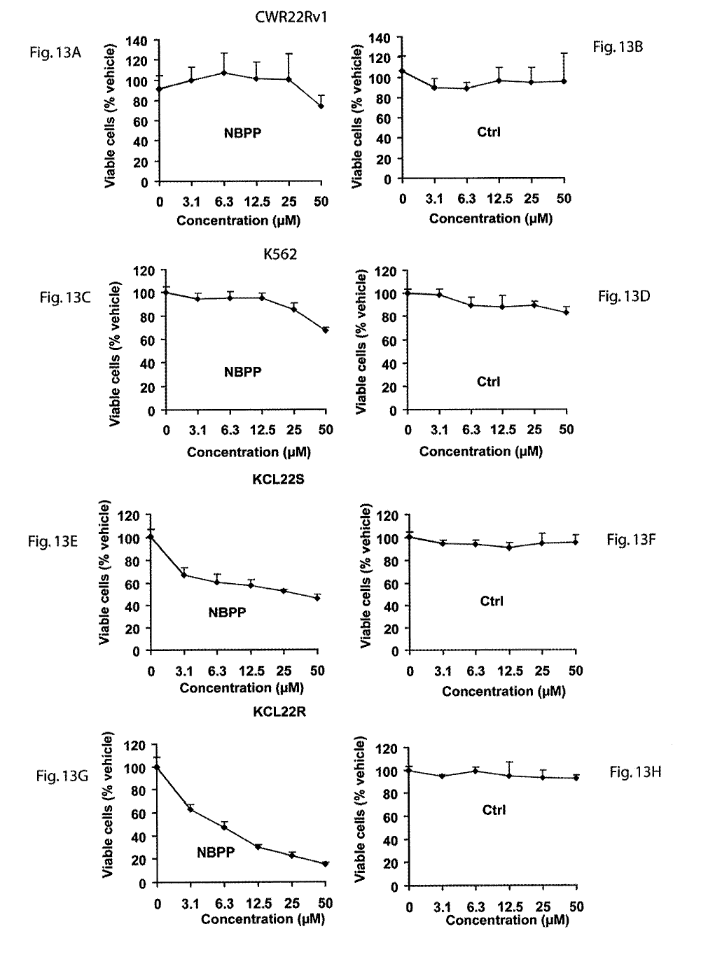

Exponentially growing CWR22Rv1 prostate cancer cells, K562 leukemia cells and imatinib-sensitive KCL22S and imatinib-resistant KCL22R chronic myeloid leukemia cells were treated with NBPP or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). The test compound decreased the number of viable prostate cancer cells (FIG. 13A) compared to the control compound (FIG. 13B). The test compound decreased the number of viable K562 cells (FIG. 13C) compared to the control compound (FIG. 13D). The test compound decreased the number of viable KCL22S cells (FIG. 13E) compared to the control compound (FIG. 13F). The test compound decreased the number of viable KCL22R cells (FIG. 13G) compared to the control compound (FIG. 13H).

### Example 16

### N⁶-benzyladenosine Inhibition of Stat5 Transcriptional Activity

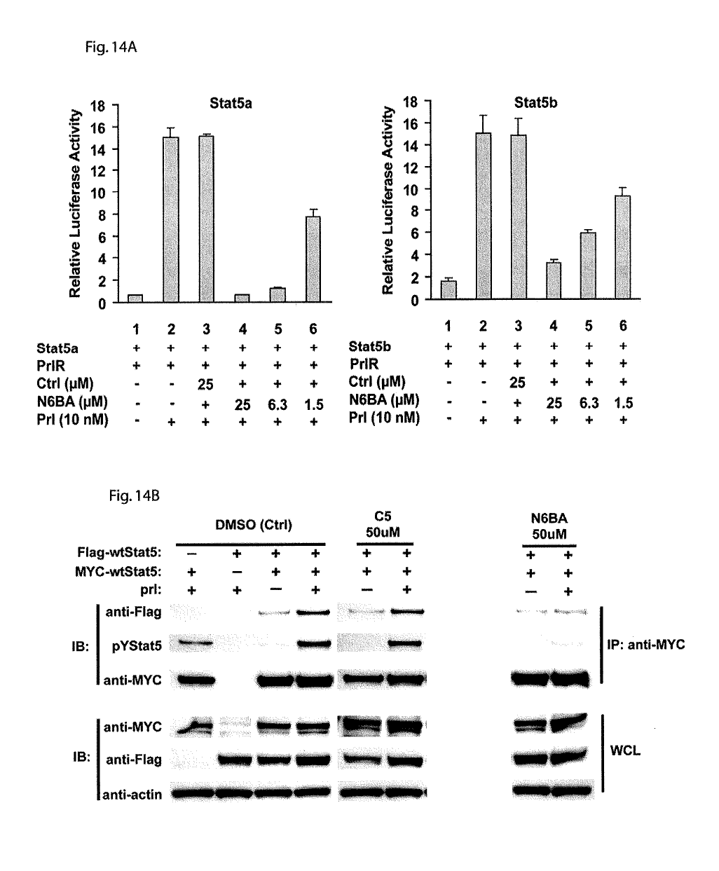

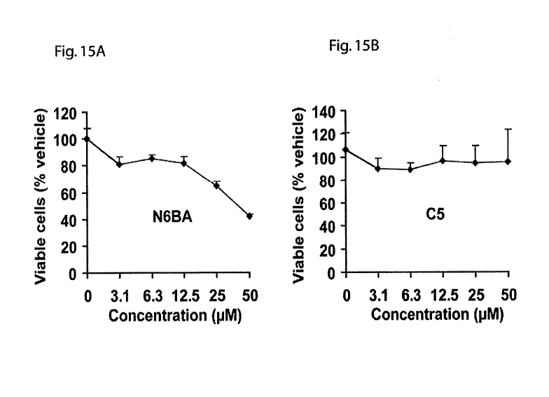

Cells of the human prostate cell line PC-3 were plated into 96-well plate at the density of 2×10⁵ per well. After 24 hours of plating, cells were transiently co-transfected using FuGENE6 (Roche) with 0.25 μg of each of pStat5a or pStat5b, pPrlR (prolactin receptor) plasmids, 0.5 μg of pBeta-casein-luc and 0.025 μg of pRL-TK (Renilla luciferase) plasmids as an internal control. After another 24 hours of transfection, the cells were starved in serum-free medium for 8 hours, pretreated with N⁶-benzyladenosine (“N6BA”), or the control compound C5, for 1 hour, and then stimulated with 10 nM human prolactin (hPrl) in the serum-free medium for additional 16 hours. The lysates were assayed for firefly and Renilla luciferase activities using the Dual-Luciferase reporter assay system (Promega). Three independent experiments were carried out in triplicate. The firefly luciferase activity was normalized to the Renilla luciferase activity of the same sample, and the mean was calculated from the parallels. From the mean values of each independent run, the overall mean and its standard deviation (S.D.) were calculated. The results, shown in FIG. 14A, demonstrate that N6BA inhibits transcriptional activity of Stat5.

### Example 17

FLAG-tagged Stat5a and MYC-tagged Stat5a were generated by standard molecular biology cloning techniques.as follows. Plasmid pCMV3-FLAG-Stat5a, pCMV3-MYC-Stat5a and pPrlR were co-transfected using FuGENE6 (Roche) into PC-3 cells (2 μg of each plasmid per 1×10⁷ cells). The cells were starved for 20 hours, pre-treated with N⁶-benzyladenosine for 2 h, then stimulated with hPrl (10 nM) in RPMI 1640 without serum for 30 minutes. The cell lysates were immunoprecipitated with 25 μl anti-FLAG M2 polyclonal affinity gel (2 μg/ml, Sigma), anti-MYC pAb (1 μg/ml, Upstate) or normal rabbit serum (NRS). The primary antibodies were used in the immunoblottings at the following concentrations: anti-FLAG pAb (1:1000; Stratagene), anti-MYC mAb (1:1000; Sigma), anti-Stat5a/b mAb (1:250) (Transduction Laboratories) detected by horseradish peroxidase-conjugated secondary antibodies.

The results are shown in FIG. 14B. Lane 1 of FIG. 14B demonstrates cells transfected only with MYC-tagged Stat5a (the third panel from the bottom). Lane 2 demonstrates cells transfected only with FLAG-tagged Stat5a (the second panel from the bottom). Lanes 3-10 demonstrate cells transfected with both FLAG-tagged Stat5a and MYC-tagged-Stat5a (the second and third panels from the bottom, respectively). Lanes 1, 2, 4, 6 and 8 represent cells stimulated with human prolactin (Prl) for 30 minutes which induces dimerization of Stat5. Two hours prior to Prl-stimulation, the cells had been treated with DMSO (lanes 1-4), the control compound C5 (lanes 5 and 6) or N6BA (lanes 7 and 8) to test whether the Stat5a/b-inhibitor compound, N6BA, would be able to inhibit dimerization of Stat5. When MYC-tagged Stat5a was immunoprecipitated from the cells (the third panel from the top) and immunoblotted with anti-FLAG pAb (top panel) the control compound did not inhibit dimerization of Stat5a (lane 6). In contrast, N6BA (lane 8) effectively inhibited Stat5a dimerization (lane 8) (=very weak band in Prl-stimulated cells). It should be noted the drug effect in this study was not due to cell apoptosis induction, as the treatment time was only 2 hours.

### Example 18

### N⁶-benzyladenosine Reduces the Viability of CWR22Rv1 Human Prostate Cancer Cells

Exponentially growing CWR22Rvl prostate cancer cells were treated with N6BA or the control compound C5 for 72 h at 3.1, 6.3. 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). The test compound decreased the number of viable prostate cancer cells (FIG. 15A) compared to the control compound (FIG. 15B).

### Example 19

### Preparation of N-(Phenylmethyl)-7-β-D-ribofuranosyl-7H-pyrrolo[2,3-d]pyrimidin-4-amine

Benzylamine (65 mg, 0.6 mmol), 4-chloro-7-β-D-ribofuranosyl-7H-Pyrrolo[2,3-d]pyrimidine (59 mg, 0.2 mmol, preparation as described in literature), diisopropylethylamine (27 mg, 0.2 mmol), and dimethylformamide (1 mL) were combined in a microwave tube (5 mL). The reaction was irradiated 15 minutes at 90° C. in the microwave. After cooling the reaction was filtered and then purified using reverse phase chromatography (gradient from 10% acetonitrile to 90% acetonitrile/ water—both solvents containing 0.1% TFA). The appropriate fractions were combined and lyophilized to yield N-(phenylmethyl)-7-β-D-ribofuranosyl-H-pyrrolo[2,3-d]pyrimidin-4-amine (30 mg, 13% yield). MS(ES⁺)=357(MH).

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1. A method of treating prostate cancer in a male in need of such treatment comprising administering to the male a therapeutically effective amount of one or more compounds of Formula I: 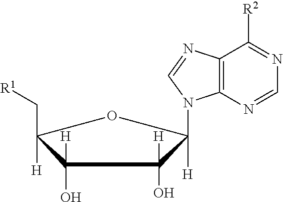
wherein:
R¹ is —OH or —O—P(O)(OH)₂; and
R² is 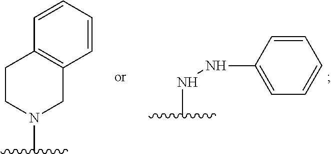 [SMILES: *N1CCc2ccccc2C1 ; *NNc1ccccc1]
or a pharmaceutically acceptable salt thereof.

2. A method of treating prostate cancer in a male in need of such treatment comprising administering to the male a therapeutically effective amount of:
N⁶-benzyladenosine,
N-(phenylmethyl)-7-β-D-ribofuranosyl-7H-pyrrolo[2,3-d]pyrimidin-4-amine,
N-(phenylmethyl)-7-β-D-ribofuranosyl-7H-pyrolo[2,3-d]pyrimidin-4-amine 5‘′-monophosphate; or
a pharmaceutically acceptable salt thereof.

3. The method according to claim 1 or 2, wherein the prostate cancer is organ-confined primary prostate cancer, locally invasive advanced prostate cancer, metastatic prostate cancer, castration-resistant prostate cancer or recurrent castration-resistant prostate cancer.

4. A method of inhibiting prostate cancer cell growth in a male comprising administering to the male in need thereof of a therapeutically effective amount of one or more compounds of Formula I: 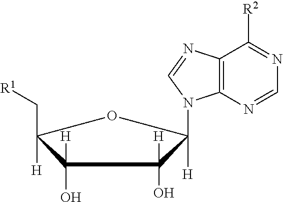
wherein:
R¹ is —OH or —O—P(O)(OH)₂; and
R² is 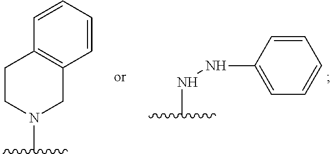 [SMILES: *N1CCc2ccccc2C1 ; *NNc1ccccc1]
or a pharmaceutically acceptable salt thereof.

5. A method of inhibiting prostate cancer cell growth in a male comprising administering to the male in need thereof of a therapeutically effective amount of one or more of:
N⁶-benzyladenosine
N-(phenylmethyl)-7-β-D-ribofuranosyl-7H-pyrrolo[2,3-d]pyrimidin-4-amine,
N-(phenylmethyl)-7-β-D-ribofuranosyl-7H-pyrrolo[2,3-d]pyrimidin-4-amine 5′-monophosphate; or
a pharmaceutically acceptable salt thereof.

6. A method of inhibiting prostate cancer cell growth comprising contacting prostate cancer cells with an effective amount of one or more compounds of Formula I: 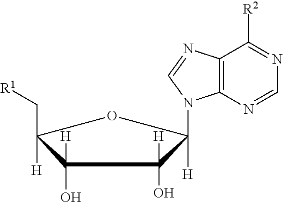
wherein:
R¹ is —OH or —O—P(O)(OH)₂; and
R² is 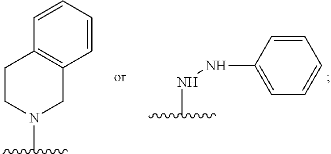 [SMILES: *N1CCc2ccccc2C1 ; *NNc1ccccc1]
or a pharmaceutically acceptable salt thereof,
effective to inhibit such cell growth.

7. A method of inhibiting prostate cancer cell growth comprising contacting prostate cancer cells with an effective amount of one or more of:
N⁶-benzyladenosine,
N-(phenylmethyl)-7-β-D-ribofuranosyl-7H-pyrrolo[2,3-d]pyrimidin-4-amine,
N-(phenylmethyl)-7-β-D-ribofuranosyl-7H-pyrrolo[2,3-d]pyrimidin-4-amine 5′-monophosphate; or
a pharmaceutically acceptable salt thereof,
effective to inhibit such cell growth.

8. A method of treating prostate cancer in a male comprising administering to a male in need of such treatment a therapeutically effective amount of:
a first agent of Formula I: 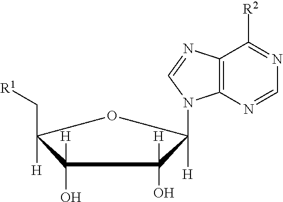
wherein:
R¹ is —OH or —O—P(O)(OH)₂; and
R² is 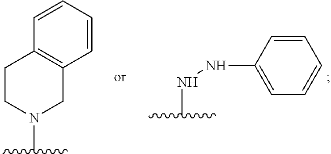 [SMILES: *N1CCc2ccccc2C1 ; *NNc1ccccc1]
or a pharmaceutically acceptable salt thereof; and
at least one of radiation therapy and chemotherapy with a second agent which is an other chemotherapeutic agent effective against prostate cancer.

9. A method of treating prostate cancer in a male comprising administering to a male in need of such treatment a therapeutically effective amount of:
a first agent selected from the group consisting of N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine 5′-monophosphate, and pharmaceutically acceptable salts thereof; and
at least one of radiation therapy and chemotherapy with a second agent which is an other chemotherapeutic agent effective against prostate cancer.

10. The method according to claim 8 or 9, wherein said second agent is selected from the group consisting of docetaxel, mitoxantrone, estramustine, doxorubicin, etoposide, vinblastine, paclitaxel, carboplatin, and vinorelbine, and combinations thereof.

11. The method according to claim 8 or 9, wherein said second agent is administered simultaneously with said first agent.

12. The method according to claim 8 or 9, wherein said second agent is administered serially with said first agent.

13. The method according to claim 12, wherein said first and second agents are administered in the same dosage form.

14. A pharmaceutical composition for treatment of prostate cancer comprising one or more compounds of Formula I: 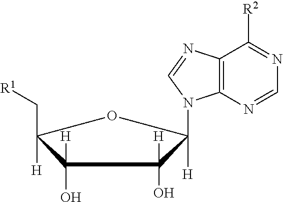
wherein:
R¹ is —OH or —O—P(O)(OH)₂; and
R² is 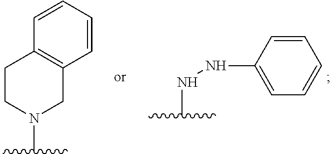 [SMILES: *N1CCc2ccccc2C1 ; *NNc1ccccc1]
or a pharmaceutically acceptable salt thereof,
and at least one other chemotherapeutic agent effective against prostate cancer.

15. A pharmaceutical composition for treatment of prostate cancer comprising N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3 -d]pyrimidin-4-amine 5′-monophosphate, or a pharmaceutically acceptable salt thereof; and at least one other chemotherapeutic agent effective against prostate cancer.

16. The composition according to claim 14 or 15, wherein said other chemotherapeutic agent is selected from the group consisting of docetaxel, mitoxantrone, estramustine, doxorubicin, etoposide, vinblastine, paclitaxel, carboplatin, and vinorelbine, and combinations thereof.

17. A method for treatment of prostate cancer comprising administering to a male in need of such treatment a therapeutically effective amount of one or more compounds of Formula I: 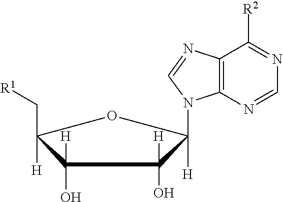
wherein:
R¹ is —OH or —O—P(O)(OH)₂; and
R² is 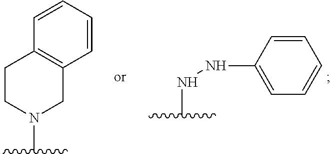 [SMILES: *N1CCc2ccccc2C1 ; *NNc1ccccc1]
or a pharmaceutically acceptable salt thereof; and
an androgen ablation therapy.

18. A method for treatment of prostate cancer comprising administering to a male in need of such treatment a therapeutically effective amount of N⁶-benzyladenosine, N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine 5′-monophosphate, or a pharmaceutically acceptable salt thereof; and an androgen ablation therapy.

19. The method according to claim 17 or 18, wherein the androgen ablation therapy comprises castration.

20. The method according to claim 17 or 18, wherein the androgen ablation therapy comprises administration of (i) at least one luteinizing hormone releasing hormone agonist, (ii) at least one anti-androgen, (iii) at least one inhibitor of prostate synthesis of androgenic steroids, or (iv) a combination of two or three of (i), (ii) and (iii).

21. A method for treating a hematological neoplasm comprising administering to a subject in need of such treatment an effective amount of at least one compound of Formula I: 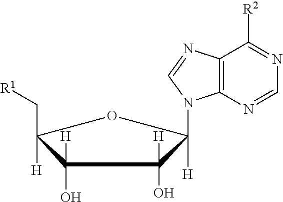
wherein:
R¹ is —OH or —O—P(O)(OH)₂; and
R² is 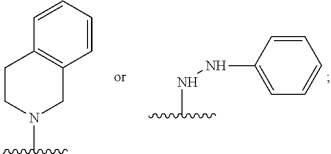 [SMILES: *N1CCc2ccccc2C1 ; *NNc1ccccc1]
or a pharmaceutically acceptable salt thereof.

22. A method for treating a hematological neoplasm comprising administering to a subject in need of such treatment an effective amount of N-(phenylmethyl)-7-β-D-ribofuranosyl-7H-pyrrolo[2,3-d]pyrimidin-4-amine, N-(phenylmethyl)-7-β-D-ribofuranosyl-7H-pyrrolo[2,3-d]pyrimidin-4-amine 5′-monophosphate, or a pharmaceutically acceptable salt thereof.

23. The method according to claim 21 or 22, wherein the hematological neoplasm is chronic myeloid leukemia, acute myelogenous leukemia or acute lymphoblastic lymphoma.

24. The method according to claim 21 or 22, wherein the hematological neoplasm is resistant to treatment with an ATP-competitive inhibitor of BCR-ABL, which resistance results from a mutation of one or more amino acid residues of BCR-ABL.

25. The method according to claim 23 wherein the hematological neoplasm is resistant to treatment with an ATP-competitive inhibitor of BCR-ABL, which resistance results from a mutation of one or more amino acid residues of BCR-ABL.

26. The method according to claim 24 wherein the ATP-competitive inhibitor of BCR-ABL is imatinib.

27. The method according to claim 25 wherein the ATP-competitive inhibitor of BCR-ABL is imatinib.

28. The method according to claim 24, wherein the mutation comprises alteration of at least one amino acid residue within the BCR ABL p-loop.

29. The method according to claim 28, wherein the mutation comprises an alteration of amino acids Tyr 253 or Glu255.

30. The method according to claim 24, wherein the mutation comprises alteration of at least one amino acid residue within the BCR-ABL activation loop.

31. The method according to claim 30, wherein the mutation comprises an alteration of His396.

32. The method according to claim 24, wherein the mutation comprises at least one mutation selected from the group consisting of F317L, H396R, M351T, H396P, Y253H, M244V, E355G, F359V, G250E, Y253F, F311L, T315I, E255V, Q252H, L387M, and E255K.

33. The method according to claim 21 or 22, further comprising administering to said individual at least one other chemotherapeutic agent effective against said hematologic neoplasm.

34. The method according to claim 33, wherein said other chemotherapeutic agent is administered serially with the compound of Formula I, the compound N-(phenylmethyl)-7-β-D-ribofuranosyl-7H-pyrrolo[2,3-d]pyrimidin-4-amine, the compound N-(phenylmethyl)-7-β-D-ribofuranosyl-7H-pyrrolo[2,3-d]pyrimidin-4-amine 5′-monophosphate, or a pharmaceutically acceptable salt thereof.

35. The method according to claim 33, wherein said other chemotherapeutic agent is administered simultaneously with the compound of Formula I, the compound N-(phenylmethyl)-7-β-D-ribofuranosyl-7H-pyrrolo[2,3-d]pyrimidin-4-amine, the compound N-(phenylmethyl)-7-β-D-riboffiranosyl-7H-pyrrolo[2,3-d]pyrimidin-4-amine 5′-monophosphate, or pharmaceutically acceptable salt thereof.

36. The method according to claim 35, wherein said other chemotherapeutic agent and the compound of Formula I, the compound N-(phenylmethyl)-7-β-D-ribofuranosyl-7H-pyrrolo[2,3-d]pyrimidin-4-amine, the compound N-(phenylmethyl)-7-β-D-ribofuranosyl-7H-pyrrolo[2,3-d]pyrimidin-4-amine 5′-monophosphate, or pharmaceutically acceptable salt thereof, are administered in the same dosage form.

37. The method according to claim 33, wherein the at least one other chemotherapeutic agent is selected from the group consisting of dasatinib, nilotinib, nelarabine, vincristine, daunorubicine, idarubicine, cytarabine, L-asparaginase, etoposide, teniposide, 6-mercaptopurine, methotrexate, cyclophosphamide, prednisone, 6-thioguanine, hydroxyurea, ATRA, ATO, fludarabine, pentostatin, cladribine, chlorambucil, bendamustine, oxaliplatin, etoposide, topotecan, azacytidine, decitabine, rituximab, alemtuzumab and ofatumumab.
